(19) United States
(12) Patent Application Publication
(10) Pub. No.: US 2011/0274617 A1
(43) Pub. Date: Nov. 10, 2011

(54) Antibody-Targeted Carrier For Contrast Agents

(75) Inventors: Abedelnasser Abulrob, Orleans (CA); Danica Stanimirovic, Greely (CA); Umar Iqbal, Ottawa (CA); Mu-Ping Nieh, Deep River (CA); John Katsaras, Deep River (CA)

(73) Assignee: NATIONAL RESEARCH COUNCIL OF CANADA, Ottawa, ON (CA)

(21) Appl. No.: 13/130,339

(22) PCT Filed: Nov. 26, 2009
(86) PCT No.: PCT/CA2009/001729

§ 371 Date: Jul. 18, 2011

Related U.S. Application Data
(60) Provisional application No. 61/118,117, filed on Nov. 26, 2008.

(51) Int. Cl. A61K 51/10; G01N 21/64; G01N 24/08; C07K 16/00; G01N 33/53; C07K 16/28; A61K 49/00; A61K 49/16; A61K 49/04; G01N 33/574; C07K 16/18

(52) U.S. Cl. 424/1.21; 435/7.23; 435/7.1; 530/391.3; 424/9.1; 424/9.341; 424/9.4; 424/9.6; 436/501

(57) ABSTRACT

A nanoconjugate is formed from a self-assembled unilamellar vesicle (ULV), at least one contrast agent which may be a MRI contrast agent, a radioisotope or a fluorophore, and at least one antibody, which may be an IgG or an antibody fragment such as a single-domain antibody. The nanoconjugate be targetted with the antibody to receptors specific to certain disease states, and thus be used in diagnostic and imaging methods using the properties o contrast agent.

### FIELD OF THE INVENTION

The present invention is directed to a composition of self-assembled, lipidic nanoparticles targeted using single domain antibodies capable of treating and imaging disease.

### BACKGROUND

Molecular imaging enables the simultaneous anatomical localization and quantitative evaluation of target biomolecules that can guide the selection of treatment protocols, whose efficacy can also be evaluated. The expected impact of these technologies in shortening the drug development cycle has been emphasized in the FDA's ‘Critical Path Initiative’ which recommends “integration of molecular and imaging biomarkers into every stage of the regulatory review for drug, diagnostic, and biologic applications” (Woodcock & Woosley, 2008).

Currently there are only a limited number of molecular imaging agents suitable for clinical applications. Most molecular imaging applications for central nervous system (CNS) diseases have been developed for radioactivity-dependent PET and SPECT modalities. These imaging compounds are typically small molecules with short circulation half-lives that can readily penetrate across the blood-brain barrier. However, similar compounds are presently lacking for the more accessible magnetic resonance imaging (MRI) modality, as well as for the rapidly developing and cheaper optical imaging modality. The clinical translation of these imaging agents will depend however, on advances in the development of new targeting/delivery moieties against disease-specific biomarkers which have been validated in animal models and the ability to scale up their production for commercialization at reasonable cost and market value.

MRI is a non-invasive and powerful medical diagnostic technique that offers high-resolution anatomical information, and is frequently used for the non-invasive detection of a variety of diseases. MRI creates images of the body using the principles of nuclear magnetic resonance. Images are usually generated using gadolinium (Gd-DTPA) as contrast agent, based on its free distribution in the body. While these images provide good anatomical information about the disease (e.g., tumor) localization and spread, MRI does not deliver adequate information about molecular characteristics of the disease (e.g., expression of certain receptors that could be targeted by drugs or transporters that may cause resistance to certain drugs, etc.), and therefore biopsy of diseased tissue and molecular analyses ex vivo (e.g., histopathology, immunochemistry, etc.) are still required.

Molecular imaging in MRI modality is currently not routinely used in clinical applications because of the lack of appropriate contrast agents that are targeted to recognize specific molecular targets. These contrast agents typically need to have very high contrast properties to provide measurable information on specific molecular target. Target characteristics are also important, including selectivity of the target for diseased tissues and the high expression/density of the target, to enable sufficient signal-to-noise ratio for detection.

While, in principle, monoclonal antibodies could be used to target contrast imaging agent or drug delivery carrier to the antigen recognition site, these antibodies are relatively large (150 kDa) proteins and can only be attached to nanoparticles in low numbers, typically less than 25 proteins per nanoparticle. Moreover, repetitive display of large proteins on the surface of nanoparticles can also be immunogenic and in some instances further accelerate biological clearance. Peptides used as a targeting moiety suffer from low affinity/specificity of binding to the target and are often prone to degradation by proteases.

Industry needs to consider systems integration approaches in order to bring together drug delivery, imaging and activation technologies into one comprehensive product. This is especially true for the CNS diseases, where delivery across the BBB imposes unique challenges for the development of both therapeutic and imaging applications. Integrated platforms for targeted drug delivery and non-invasive monitoring and quantification of drug distribution and accumulation/release at desired targets by means of imaging are currently not available.

Phospholipid bilayers forming spherical unilamellar vesicles (ULVs), or liposomes, could be used as biodegradable or biocompatible drug carriers to enhance the potency and reduce the toxicity of therapeutics. Typically, ULVs are produced by sonication or high-pressure multi-stage extrusion of multi-lamellar vesicles (MLV). The major drawbacks of these methods include degradation and modification of phospholipids (e.g. oxidation, hydrolysis, denaturation), difficulties in producing single size population liposomes, and low throughput. ULV produced by sonication and extrusion methods are inherently unstable (not thermodynamically stable) and may, over time, revert to MLVs.

ULVs are also capable of entrapping and delivering contrast imaging agents. There are two categories of liposomal contrast agents: a) those that entrap paramagnetic molecules in the aqueous compartment and b) those that incorporate these molecules in the liposomal membrane, either by covalent attachment to the lipid acyl chains or by chelation to a ligand which is incorporated into the membrane. With regard to MRI, incorporating the contrast agent within the outer or inner membrane is preferred, as the bound paramagnetic ions possess a much longer rotational correlation time and therefore have a greater relaxivity/mole than those in solution (i.e. better contrast).

Gd-DTPA is an FDA approved imaging contrast agent for MRI. To achieve sufficient signal-to-noise ratio and detectable signal for molecular imaging applications, the concentration of Gd at diseased molecular recognition sites has to be very high; in other words, a single Gd molecule per targeting moiety is insufficient to achieve detectable signal.

To accelerate approval of molecular imaging agents for clinical use, it is desirable to have the ability to assess them using multi-modal imaging, for example optical in animal studies and MRI in animal and human studies.

### SUMMARY OF THE INVENTION

The present invention comprises a composition of self-assembled, lipidic nanoparticles targeted using single domain antibodies, and capable of treating and imaging disease.

The present invention comprises novel formulations of lipid-based spontaneously forming nanoparticles. These nanoparticles may comprise spontaneously forming unilamellar vesicles (ULVs) comprising phospholipids, and may be used for noninvasive molecular imaging. Such liposomal-based delivery systems may display efficacy and commercial viability via: a) vesicle stability i.e., extended shelf life; b) well-defined, monodisperse ULV size; c) extended plasma half-life in vivo, and d) potential for scale-up to industrial sized production.

The present invention provides a nanoconjugate comprising:
(a) a self-assembled unilamellar vesicle (ULV);
(b) at least one contrast agent; and
(c) at least one antibody.

The ULV may be comprised of dimyristoyl phosphatidylcholine (DMPC); dihexanoyl phosphatidylcholine (DHPC); dimyristoyl phosphatidylglycerol (DMPG); and distearoyl phosphoethanolamine-[maleimide(polyethylene glycol)-2000] (DSPE-PEG-maleimide).

In the nanoconjugate described above, the contrast agent may be a MRI contrast agent, a radioisotope, a fluorophore, or a combinations thereof. In one embodiment, the contrast agent may be a MRI agent, and may be gadolinium-diethylene-triamine-pentaacetic acid bis-oleate (Gd-DTPA-BOA). In another embodiment, the fluorophore may be Cy5.5.

In the nanoconjugate described above, the antibody may specifically bind an epitope present in the brain endothelial cells or tumor cells. For example, the antibody may selectively bind Epidermal Growth Factor Receptor (EGFR). In another example, the antibody may selectively bind Insulin-like Growth Factor Binding Protein 7 (IGFBP7). In one specific example, the antibody may comprise complementarity determining region (CDR) sequences RTSRRYAM or RTFSRLAM (CDR1; SEQ ID NOs:1 and 2), GISRSGDGTHYAYSV (CDR2; SEQ ID NO:3), and AAARTAFYYYGNDYNY (CDR3; SEQ ID NO:4). Alternatively, the antibody may comprise the sequence:

(SEQ ID NO: 5)
AIAIAVALAGFATVAQAQVKLEESGGGLVQAGGSLRLSCAASGRTSRRYA
MGWFRQAPGKEREFVAGISRSGDGTHYAYSVKGRFTISRDNAANTVELQM
NSLKPEDTAVYFCAAARTAFYYYGNDYNYWGQGTQVTVSS,

or a sequence substantially identical thereto. In another alternative, the antibody may comprise the sequence:

(SEQ ID NO: 6)
AIAIAVALAGFATVAQAQVKLEESGGGSVQPGGSLRLSCAASGRTFSRL
AMGWFRQAPGKERELVAGISRSGDGTHYAYSVKGRFTISRDNAANTV
ELQMNSLKPEDTAVYFCAAARTAFYYYGNDYNYWGQGTQVTVSS,

or a sequence substantially identical thereto.

The present invention further provides a method of forming unilamellar vesicles (ULV) incorporating at least one contrast agent, the method comprising:
(a) mixing dimyristoyl phosphatidylcholine (DMPC); dihexanoyl phosphatidylcholine (DHPC); dimyristoyl phosphatidylglycerol (DMPG); distearoyl phosphoethanolamine-[maleimide(polyethylene glycol)-2000] (DSPE-PEG-maleimide) and gadolinium-diethylene-triamine-pentaacetic acid bis-oleate (Gd-DTPA-BOA); and
(b) allowing the spontaneous formation of ULV.

In the method as described above, an antibody may be bioconjugated to DSPE-PEG-maleimide prior to step (a), thus incorporating the antibody into the nanoconjugate.

The present invention also provides a method for in vivo imaging of cells or tissues in a mammal, the method comprising the steps of:
(a) administering to the mammal a composition comprising the nanoconjugate described herein, wherein the antibody is specific for a selected receptor;
(b) waiting a time sufficient to allow the antibody to bind to the selected receptor; and
(c) imaging the cells or tissues with a non-invasive imaging technique whose resolution is enhanced by the presence of the particles on or within the cells.

The imaging technique used may be selected from the group consisting of magnetic resonance imaging, magnetic spectroscopy, X-ray, positron emission tomography, optical imaging, computed tomography, and ultrasonic imaging. The method as described may allows for imaging of one or more tumors, metastases, vascularized malignant cell clusters, or individual malignant cells selected from the group consisting of brain cancer, colon cancer, breast cancer, prostate cancer, lung cancer, pancreatic cancer, endometrial cancer, oral cancer, liver cancer, and renal cancer or any other cancer.

In one embodiment of the method as described above, the selected receptor may be is specifically expressed by tumor endothelial cells. The selected receptor may be IGFBP7, and the antibody may be as described above. In another embodiment, the selected receptor may be EGFR.

In one aspect, the invention comprises a method for detecting glioblastoma in a patient, comprising:
(a) contacting a tissue of interest with a nanoconjugate as described herein, wherein the antibody is specific for the IGFBP7 or EGFR, and may comprise the specific antibodies described herein; and
(b) measuring the level of binding of the nanoconjugate, wherein an elevated level of binding, relative to normal tissue, is indicative that the tissue is neoplastic.

In yet another aspect, the present invention provides a method for detecting a tissue expressing IGFBP7, comprising:
(a) contacting a tissue of interest with a nanoconjugate as described herein, wherein the sdAb is specific for the IGFBP7, and may comprise the specific antibodies described herein; and
(b) measuring the level of binding of the nanoconjugate, wherein an elevated level of binding, relative to normal tissue is indicative of the presence of a tumor expressing IGFBP7.

In yet another aspect, the present invention provides a method for detecting a tissue expressing EGFR, comprising:
(a) contacting a tissue of interest with a nanoconjugate as described herein, wherein the antibody is specific for the EGFR, and may comprise the specific antibodies described above; and
(b) measuring the level of binding of the nanoconjugate, wherein an elevated level of binding, relative to normal tissue is indicative of the presence of a tumor expressing EGFR.

In one embodiment, the step of measuring is performed by magnetic resonance imaging. The nanoconjugate used in the method as just described may further comprise a fluorescent imaging agent, and the step of measuring may be performed using fluorescence imaging.

In another aspect, the invention comprises a method for determining the location of glioblastoma brain tumor cells in a patient pre-operatively, intra-operatively, and/or post-operatively, comprising administering a composition comprising a nanoconjugate as described herein, wherein the antibody is specific for the IGFBP7 or EGFR, and may comprise the specific antibodies described above, and a pharmaceutically acceptable carrier to the patient, wherein the composition is administered in an amount sufficient to image glioblastoma cells in vivo; and
(a) pre-operatively measuring the level of binding of nanoconjugate by magnetic resonance imaging to determine the location of glioblastoma cells, wherein an elevated level of binding, relative to normal tissue, is indicative of the presence of glioblastoma cells;
(b) intra-operatively measuring the level of binding of the nanoconjugate by fluorescence imaging to determine the location of residual glioblastoma cells, wherein an elevated level of binding, relative to normal tissue, is indicative of the presence of residual glioblastoma cells;
(c) post-operatively measuring the level of binding of the nanoconjugate by magnetic resonance imaging to determine the location of glioblastoma cells, wherein an elevated level of binding, relative to normal tissue, is indicative of the presence of tumor cells; or
(d) a combination of (a), (b) or (c).

In yet another aspect, the invention comprises a method for in vitro detection or quantification of biological or chemical molecule in a sample is also provided by the present invention. The method comprises the steps of:
(a) contacting the sample with a solution comprising the nanoconjugate of the present invention, so as to form a complex between the molecule and the nanoconjugate; and
(b) detecting and/or quantifying said complex formed.

The step of detecting and/or quantifying may be performed by magnetic resonance imaging, fluorescence imaging, or a combination thereof.

The naonconjugates of the present invention may be used as a MRI contrast agent, as they contain a very high number of Gd molecules, up to 60,000 Gd molecules per ULV; this may result in increased sensitivity (i.e., high number of contrast agent molecules at antigen recognition sites) and increased signal-to-noise ratio. The ULVs may be self-assembled using components loaded with Gd-DTPA-BOA, thereby achieving high numbers of Gd molecules carried by each ULV.

The nanoconjugates of the present invention may also be used in bi-modal imaging for MRI and optical in vivo imaging. In one embodiment, the bi-modal capacity is introduced by attaching an optical probe such as the near-infrared probe, Cy5.5, applicable to in vivo optical imaging, to PEG moieties incorporated into self-assembled Gd-loaded ULVs

The present invention provides a process for formulating highly stable, self-assembled monodisperse, nanoscopic ULVs composed of commonly available low cost phospholipids; the ULVs can be tailored to suit a variety of biomedical needs. Self-assembled ULVs have advantages over ULVs formed by sonication or extrusion in key areas important for development of commercially viable drug delivery formulations: namely a) both their size and entrapment efficiency can be controlled during the self assembly process; b) they are very stable (long shelf life and in vivo); and c) the process can be easily scaled up for manufacturing.

The present invention also produces targeted drug delivery/imaging formulations. Bioconjugation of antibodies, and particularly antibody fragments such as sdAbs, to ULVs may enhance sensitivity and specificity of targeting of imaging/drug delivery formulation. Compared to the conventional 150 kDa IgG molecules, a larger number of antibody fragments, such as sdAbs, can be incorporated into ULVs, lending polyvalency and increasing avidity. Moreover, antibody fragments/sdAbs may be more stable and soluble compared to conventional antibodies

The ability to perform these molecular analyses non-invasively by in vivo imaging by MRI at the time of diagnosis and during disease treatment may greatly improve treatment efficacy by a) obtaining early molecular information on disease, b) adjusting treatment to fit ‘personal’ characteristics of disease, c) selecting appropriate patient populations for clinical trials.

### BRIEF DESCRIPTION OF THE DRAWINGS

In the drawings, like elements are assigned like reference numerals. The drawings are not necessarily to scale, with the emphasis instead placed upon the principles of the present invention. Additionally, the embodiments depicted are but some of a number of possible arrangements utilizing the fundamental concepts of the present invention. The drawings are briefly described as follows:

FIG. 1 is a schematic representation of one embodiment of a unilamellar vesicle (ULV) functionalized with single domain antibody and loaded with gadolinium (Gd) and optical imaging contrast (Cy5.5) in the hydrophobic shell and with the drug in the hydrophilic core. DMPC=Dimyristoyl phosphatidylcholine; DMPG=Dimyristoyl phosphatidylglycerol; DHPC=Dihexanoyl phosphatidylcholine; Gd-DPTA BOA=Gadolinium diethylenetriaminopentaacetic acid bisoleate; PEG-DSPE=Distearoyl Phosphoethanolamine-N-[Methoxy(Polyethylene glycol)-2000]; amine-PE=Dipalmitoyl Phosphoethanolamine-N-(dodecanylamine). Dode-PE=Dipalmitoyl Phosphoethanolamine-N-(dodecanylamine).

FIG. 2 graphically shows LC-Gd ULV nanoparticle size determined by dynamic light scattering.

FIG. 3 graphically shows the size distribution of spontaneously formed ULVs formulated with HC-Gd as determined by dynamic light scattering.

FIG. 4 shows evaluation of LC-Gd nanoparticles at various total lipid concentrations: 10.0 (triangles; darkest grey), 5.0 (diamonds; lightest grey), 1.0 (squares; black), 0.2 (circles; 2^{nd} lightest grey) wt %, using Small Angle Neutron Scattering (SANS). The peaks at ˜0.055 and ˜0.11 Å⁻¹ correspond to the first and second order reflections from an MLV.

FIG. 5 shows evaluation of HC-Gd nanoparticles using SANS. Total lipid concentrations are: 10.0 (triangles; darkest grey), 5.0 (diamonds; lightest grey), 1.0 (squares; black), 0.2 (circles; lightest grey) wt %. The peaks at ˜0.055 and ˜0.11 Å⁻¹ correspond to the first and second order reflections from MLVs. The peak intensity shows that, compared to the 20 mol % Gd sample, the 40 mol % sample results in more MLV being formed.

FIG. 6 shows SANS data of the HC-Gd 20 nm mixture with total lipid concentrations of 1.0 (black) and 0.5 (grey) wt % annealed at 50° C. for 18 hours (inverted triangles) and 3 days (triangles). The nanoparticle mixtures were reformulated by replacing PEGylated-DSPE-maleimide with PEGylated-DSPE-amine and DMPC with DMPG (in FIG. 4). The MLV peaks disappeared indicating the absence of MLVs. The gray curves are the best fits to the data using the ellipsoidal shell model. The data and fits to the data are resealed for viewing clarity.

FIG. 7 is a schematic representation of an ellipsoidal shell model.

FIG. 8 shows a table of the measurement of the Gd molecules (ng/ml) in ULV nanoparticle formulations (LC- and HC-Gd) determined using ICP-MS.

FIG. 9 shows imaging of EGFR-expressing subcutaneous xenograft tumors in nude mice using Gd-Cy5.5-ULVs (40 mol % Gd) (A) or Gd-Cy5.5-ULVs (HC-Gd) targeted with the monoclonal IgG antibody C225 against EGFR (B). Images were taken 24 h post-injection. Cy5.5 fluorescence was detected only in the tumor xenograft of animals injected with targeted ULV but not in animals injected with non-targeted ULVs.

FIG. 10 shows imaging of time-dependent tumor accumulation of C225-targeted (A) vs. non-targeted (B) Gd-Cy5.5-ULVs (HC-Gd) in EGFR expressing subcutaneous flank xenograft tumors in nude mice. Cy5.5 fluorescence was detected only in the tumor xenograft of animals injected with C225 Ab targeted ULV but not in animals injected with non-targeted ULVs.

FIG. 11 shows quantitation of time-dependent in vivo accumulation of C225-targeted vs. non-targeted Gd-Cy5.5-ULVs (HC-Gd) in EGFR-expressing subcutaneous flank xenograft tumors in mice (from FIG. 10).

FIG. 12 shows imaging (whole body dorsal scan) of in vivo biodistribution (24 h after injection) of non-targeted (A) and C225-targeted (B) Gd-Cy5.5-ULVs vesicle (HC-Gd) in tumor-bearing mice. Cy5.5 fluorescence was detected only in the tumor xenograft of animals injected with C225 Ab targeted ULV but not in animals injected with non-targeted ULVs.

FIG. 13 show imaging (whole body ventral scan) of in vivo biodistribution (24 h after injection) of non-targeted (A) and C225-targeted (B) Gd-Cy5.5-ULVs vesicle (HC-Gd) in tumor-bearing mice. Cy5.5 fluorescence was detected only in the tumor xenograft of animals injected with targeted ULV but not in animals injected with non-targeted ULVs.

FIG. 14 shows ex vivo imaging of excised tumor and skeletal muscle 24 h after injection of C225-targeted (A) or nontargeted (B) Gd-Cy5.5 ULVs (HC-Gd). Cy5.5 fluorescence was detected only in the excised tumor of animals injected with C225 Ab targeted ULV but not in excised muscle of similar size. Animals injected with non-targeted ULVs had minimal fluorescence in both excised tumor and muscle.

FIG. 15 shows the presence of Cy5.5 fluorescence (red) in tumor section immunostained for EGFR (green) from mice injected with C225-targeted (A) or non-targeted (B) Gd-Cy5.5-ULVs (HC-Gd). Cy5.5 fluorescence was detected only in the tumor sections of animals injected with targeted ULV but not in animals injected with non-targeted ULVs.

FIG. 16 shows gadolinium concentration measurement using laser ablation ICP-MS in tumors excised from mice injected with C225-targeted or non-targeted Gd-Cy5.5-ULVs (HC-Gd). N=14 per group. High number of Gd was measured in C225 monoclonal targeted ULV in tumor compared to low content of Gd in non-targeted ULV.

FIG. 17 shows measurement of the gadolinium content in organs using ICP-MS in tumors and organs excised from mice injected with C225-targeted Gd-Cy5.5-ULVs (HC-Gd) in Table (A) and graph (B and C) form. FIG. 17C shows the concentration of Gd in ng/mg or ppm, and the measurement of Gd content in organs relative to dry weight of the organ. High number of Gd was measured in C225 monoclonal targeted ULV in tumor compared to low content of Gd in non-targeted ULV.

FIG. 18 shows optical in vivo imaging of the head of the mice bearing an orthotopic brain tumor at different time points after the injection of either non-targeted (A) or IGFBP7 sdAb-targeted (B) Gd-Cy5.5-ULVs (HC-Gd 20 nm). Cy5.5 fluorescence was detected only in the brain tumor of animals injected with IGFBP7 sdAb targeted ULV but not in animals injected with non-targeted ULVs.

FIG. 19 shows depth-concentration analysis (A) and volumetric analysis (B) of head imaging after injection of non-targeted or IGFBP7 sdAb-targeted Gd-Cy5.5-ULVs vesicle HC-Gd 20 nm) in orthotopic brain tumor-bearing mice (from FIG. 18). Two way ANOVA was run to test significance.

FIG. 20 shows biodistribution of non-targeted (A) and IGFBP7 sdAb-targeted (B)Gd-Cy5.5-ULVs vesicles (HC-Gd 20 nm) 24 h after injection into orthotopic brain tumor-bearing mice by whole-body in vivo optical imaging (dorsal scan). Two examples of each are shown. Cy5.5 fluorescence was detected only in the brain tumor of animals injected with targeted ULV but not in animals injected with non-targeted ULVs.

FIG. 21 shows biodistribution of non-targeted (A) and IGFBP7 sdAb-targeted (B) Gd-Cy5.5-ULVs vesicles (HC-Gd 20 nm) 24 h after injection into orthotopic brain tumor-bearing mice by ex vivo optical imaging of excised organs. Cy5.5 fluorescence was detected in the brain tumor of animals injected with targeted ULV but not in animals injected with non-targeted ULVs. High signal was detected non-specifically in liver.

FIG. 22 shows ex vivo imaging of brain tumors 24 h after injection of Gd-Cy5.5-ULVs (HC-Gd 20 nm) non-targeted (A) or targeted with the anti-IGFBP7 sdAb (B) that recognize brain tumor vasculature. Cy5.5 fluorescence was detected only in the implanted brain tumor of animals injected with targeted ULV but not in animals injected with non-targeted ULVs.

FIG. 23 shows the effect of Gd-DTPA-BOA ULVs (HC-Gd 20 nm) on T1 relaxation measured by 9.4 T MRI. The inset shows the location of different samples in the phantom apparatus.

FIG. 24A shows MRI in vivo imaging of the head in orthotopic brain tumor bearing mice injected with non-targeted Gd-Cy5.5-ULVs vesicle (HC-Gd 20 nm). FIG. 24B shows the subtraction images of FIG. 24A.

FIG. 25A shows another example of MRI in vivo imaging of the head in orthotopic brain tumor-bearing mice injected with non-targeted Gd-Cy5.5-ULVs vesicle (HC-Gd 20 nm).

FIG. 25B shows the subtraction images of FIG. 25A.

FIG. 26A shows MRI in vivo imaging of the head in orthotopic brain tumor bearing mice injected with IGFBP7 single domain antibody-targeted Gd-Cy5.5-ULVs vesicle (HC-Gd 20 nm). FIG. 26B shows the subtraction images of FIG. 26A.

FIG. 27A shows another example of MRI in vivo imaging of the head in orthotopic brain tumor bearing mice injected with IGFBP7 single domain antibody-targeted Gd-Cy5.5-ULVs vesicle (HC-Gd 20 nm). FIG. 27B shows the subtraction images of FIG. 27A.

FIG. 28 shows pharmakinetics analysis of unilamellar vesicles labelled with Cy5.5 (40 mol % Gd with 20 nm size) and injected intravenously in normal CD1 mice. Blood samples were taken at different time points and fluorescence was measured using a fluorescence plate reader. Analysis was undertaken using WinNonlin professional software using one-compartment, bolus injection modeling. R2=0.9932; Plasma half-life=96.83 minutes, Vss (Apparent volume of distribution)=1.004 ml; MRT (mean residence time)=139.7 min, CL (Clearance)=0.00718 molecules/min.

### DETAILED DESCRIPTION OF PREFERRED EMBODIMENTS

The present invention is directed to a composition of self-assembled, lipidic nanoconjugates targeted using single domain antibodies capable of treating and imaging disease.

When describing the present invention, all terms not defined herein have their common art-recognized meanings. To the extent that the following description is of a specific embodiment or a particular use of the invention, it is intended to be illustrative only, and not limiting of the claimed invention. The following description is intended to cover all alternatives, modifications and equivalents that are included in the spirit and scope of the invention, as defined in the appended claims.

One embodiment of the present invention comprises antibody-modified ULVs formulated to incorporate both gadolinium ions and fluorescent dyes, and be capable of selectively targeting disease affected sites in the brain or in tumors, where the anatomical localization and molecular characteristics of diseased cells can be elucidated using MRI or optical imaging, or both simultaneously or consecutively. The ULVs also provide a vehicle by which a therapeutic can be delivered to these diseased sites in the same formulation, enabling a non-invasive monitoring of both therapeutic delivery and therapeutic efficacy.

In one embodiment, the present invention provides a nanoconjugate comprising:
(a) a self-assembled unilamellar vesicle (ULV);
(b) at least one contrast agent; and
(c) at least one antibody.

By the term “self-assembled unilamellar vesicle” or “spontaneously-formed unilamellar vesicle”, it is meant spontaneously formed, homogenous monodisperse, and size-controlled ULVs. These ULVs may be tailored to suit a variety of biomedical and nutraceutical needs, at the same time being suitable for industrial scale production. The self-assembled ULV may have advantages over ULVs formed by sonication or extrusion. For example, their size and entrapment efficiency can be controlled during the self assembly process, they are very stable, and the process for producing them can be easily scaled up for manufacturing.

The ULV may comprise any suitable lipids known to form ULVs. For example, and the ULV may comprise lipids including, but not limited to dimyristoyl phosphatidylcholine (DMPC); dimyristoyl phosphatidylglycerol (DMPG); dihexanoyl phosphatidylcholine (DHPC); distearoyl Phosphoethanolamine-N-[Methoxy(Polyethylene glycol)-2000] (PEG-DSPE); dipalmitoyl Phosphoethanolamine-N-(dodecanylamine) (amine-PE); and dipalmitoyl phosphoethanolamine-N-(dodecanylamine) (Dode-PE).

As would be understood by those of skill in the art, the composition of the ULV may vary based on the type of contrast agent and/or sdAb to be included in the nanoconjugate, as well as the type of application for which the nanoconjugate is to be used. For example, and without wishing to be limiting in any manner, the amount of each lipid in a composition of ULVs may independently be between about 0 and 55 mol. % DMPC; between about 20 and 30 mol. % DHPC; between about 0 and 35 mol. % DMPG; between about 3 and 10 mol. % DSPE-PEG-maleimide; and between about 0 and 1 mol. % dode-PE. In specific, non-limiting examples, the amount of each lipid may independently be 0, 30.6, or 50.5 mol. % DMPC; 23.8 or 23.9 mol. % DHPC; 0.4, 0.5, or 31.2 mol. % DMPG; 5 mol. % DSPE-PEG-maleimide; 0 or 0.1 mol. % Dode-PE.

Without wishing to be limiting, the ULV may comprise, for example:
(a) DMPC=30.6 mol. %; DHPC=23.9 mol. %; DMPG=0.4 mol. %; DSPE-PEG-maleimide=5 mol. %; dode-PE=0.1 mol. %;
(b) DMPC=50.5 mol. %; DHPC=23.9 mol. %; DMPG=0.5 mol. %; DSPE-PEG-maleimide=5 mol. %; dode-PE=0.1 mol. %; or
(c) DHPC=23.8 mol. %; DMPG=31.2 mol. %; and DSPE-PEG-amine.

As would be understood by one of skill in the art, other combinations of lipids are encompassed by the present invention. Various physical parameters may also aid in determining the ULV composition. Such parameters include, but are not limited to charge density, chain length, temperature, and salt concentration. The skilled artisan will be adept in adapting the ULV composition to account for such parameters.

In addition to the amounts of lipids described above, the ULVs may be characterized by ratios of certain lipid components. For example, one embodiment of the ULVs may possess a constant long-to-short chain lipid ratio of 3.0-5.0; and/or a constant DSPE-PEG2000-Maleimide/total lipid of less than 0.05. In one embodiment, when Gd-DTPA-BOA is used (see below), the ULV may also comprises a DMPC/DMPG ratio of 100-0.

It is noted that, in certain examples of ULVs described above, a portion of the phospholipids incorporate PEG molecules. Without wishing to be bound by theory, PEG molecules incorporated on the surface of the ULV nanoparticle may create a formulation that is not readily recognized or cleared by the reticuloendothelial system, therefore improving their plasma stability and plasma half-life. Such “stealth” formulations may optimize the blood circulation half-life. PEG molecules may be covalently attached to a phospholipid (prior to ULV assembly) by methods well-known to those of skill in the art.

While DSPE-PEG2000-Maleimide is mentioned above as a specific non-limiting example, any suitable size PEG may be used, and is encompassed by the present invention. Without wishing to be limiting, the PEG may be in the range of about 1000 to 5000 Da; for example, the PEG may be about 1000, 1250, 1500, 1750, 2000, 2250, 2500, 2750, 3000, 3250, 3500, 3750, 4000, 4250, 4500, 4750, or 5000 Da, or any size therebetween.

The ULVs may vary in size, based on their composition and/or other variables. For example, the ULV may generally be between about 30 and 150 nm; for example, the ULV may be about 30, 40, 50, 60, 70, 80, 90, 100, 110, 120, 130, 140, or 150 nm, or any size therebetween.

The nanoconjugate of the present invention also comprises at least one contrast agent. The contrast agent may be a MRI contrast agent, an optical imaging agent, or a combination thereof.

The nanoconjugate of the present invention may comprise a MRI contrast agent. The MRI contrast agent may be any MRI contrast agent suitable for incorporation into ULVs. The MRI contrast agent should preferably be an agent that produces T1 enhancement effect, and should be preferably incorporated into the ULV with minimal effect of the morphology of the vesicle. For example, the MRI contrast agent may be, but is not limited to a chelated paramagnetic ion. For example, the paramagnetic ion may be gadolinium, manganese, ytterbium, europium, or the like. In a specific, non-limiting example, the paramagnetic ion may be a gadolinium (Gd) ion.

Any paramagnetic ion-based lipid that may be incorporated into the lipid bilayer of the ULV would be suitable for use in the present invention. Different chelating agents or alternatives may also be used, such a, but not limited to EDTA, DTPA and DOTA and the like. For example, the chelating agent may be coupled directly to a lipid such as, but not limited to phosphatidyl ethanolamine, bis-oleate, and the like, or through linking groups. In a specific, non-limiting embodiment, the contrast agent incorporated into the ULV may comprise gadolinium-diethylene-triamine-pentaacetic acid bis-oleate (Gd-DTPA-BOA).

The MRI contrast agent may be incorporated into the ULV at a molar ratio in the range of 15 to 40 mol % of MRI contrast agent to total lipid mixture. For example, and without wishing to be limiting, the molar ratio may be 15, 20, 25, 30, 35, or 40 mol %, or any value therebetween. In a specific, non-limiting example, the MRI contrast agent may be incorporated into the ULV at a molar ratio of 20 mol % or 40 mol %.

As the MRI contrast agent may be incorporated directly into the ULV, a high number of the contrast agent molecules may be incorporated into the nanoconjugate of the present invention. Without wishing to be bound by theory, the inclusion of a high number of contrast agent molecules into the nanoconjugate may result in increased sensitivity (i.e., high number of contrast agent molecules at the site of interest) and increased signal-to-noise ratio. For example, the number of paramagnetic ions per ULV may be in the range of about 5,000 to about 60,000; for example, the ULV may comprise about 5000, 10,000, 15,000, 20,000, 25,000, 30,000, 35,000, 40,000, 45,000, 50,000, 55,000, or 60,000, or any amount therebetween, paramagnetic ions per ULV. In a specific, non-limiting example, the ULV may comprise up to 60,000 Gd molecules per ULV. Without wishing to be bound by theory, ULVs exhibiting high Gd payload show enhanced T1 effect in 9.4 T MRI phantoms, comparable to the clinically used Gd-DTPA—Magnevist.

The contrast agent in the nanoconjugate may further comprise one or more than one optical imaging agent, thus creating a bimodal imaging agent. The optical imaging agent may be, for example, but not limited to, a radioisotope or a fluorophore. For example, the optical imaging agent may be, but is not limited to Cy5.5, Cy7, Cy7.5. Alexa 680, Alexa 750, ICG, IR800, or any fluorophore that emits between 650 nm and 900 nm. Multiple copies of the same or different optical imaging agent may be present in the nanoconjugate. The optical imaging agent may be incorporated into the ULV by conjugation to a PEG molecule.

The nanoconjugate of the present invention further comprises at least one antibody (Ab) as a targeting moiety. By the term “antibody”, it is meant any suitable antibody; for example, but not limited to antibodies (such as IgG) and antibody fragments, whether naturally-occurring or recombinantly-produced. The antibody may be engineered by molecular techniques, and may comprise associated sequences (such as signal peptides, purification tags, etc). Antibody fragments may comprise, but are not limited to Fab, Fab′, Fv, scFv, and single-domain antibodies.

By the term “single-domain antibody” or “sdAb”, it is meant an antibody fragment comprising a single protein domain. Single domain antibodies may comprise any variable fragment, including V_{L}, V_{H}, V_{H}H, V_{NAR}, and may be naturally-occurring or produced by recombinant technologies. For example V_{H}s, V_{L}s, V_{H}Hs, V_{NAR}s, may be generated by techniques well known in the art (Holt, et al., 2003; Jespers, et al., 2004a; Jespers, et al., 2004b; Tanha, et al., 2001; Tanha, et al., 2002; Tanha, et al., 2006; Revets, et al., 2005; Holliger, et al., 2005; Harmsen, et al., 2007; Liu, et al., 2007; Dooley, et al., 2003; Nuttall, et al., 2001; Nuttall, et al., 2000; Hoogenboom, 2005; Arbabi-Ghahroudi et al., 2008). In the recombinant DNA technology approach, libraries of sdAbs may be constructed in a variety of ways, “displayed” in a variety of formats such as phage display, yeast display, ribosome display, and subjected to selection to isolate binders to the targets of interest (palming). Examples of libraries include immune libraries derived from llama, shark or human immunized with the target antigen; non-immune/naïve libraries derived from non-immunized llama, shark or human; or synthetic or semi-synthetic librairies such as V_{H}, V_{L}, V_{H}H or V_{NAR} libraries.

The small size of the sdAbs allow their conjugation in nanoconjugates with a much higher binding site density compared to larger antibody fragments (>5 fold compared to IgGs and 2-fold compared to scFvs) and do not promote nanoparticle aggregation associated with scFvs and IgGs, resulting in much more active nanoconjugates, and more robust signal amplification strategy. Higher levels of imaging signal per unit level of target-probe interaction lead to higher sensitivity for any particular imaging modality. Additionally, the highly stable nature of sdAbs allows for flexibility in terms of choosing optimal conjugation chemistry conditions (Huang et al, 2007), leading to a more active end product.

In one embodiment, the antibody may be a sdAb that recognizes and binds to an antigen present in tumor endothelial cells. For example, and without wishing to be limiting in any manner, the single domain antibody may selectively bind Insulin-like Growth Factor Binding Protein 7 (IGFBP7), which is strongly upregulated in vessels of glioblastoma tumors undergoing neovascularization. This target is less expressed in vessels of low grade gliomas. Without wishing to be limiting in any manner, the single domain antibody may be an sdAb as described in PCT/CA2009/001460 entitled “Formulations Targetting IGFBP7 for Diagnosis and Therapy of Cancer”, the disclosure of which is incorporated herein by reference where permitted. In a specific, non-limiting example, the sdAb may comprise complementarity determining region (CDR) sequences RTSRRYAM [SEQ ID NO. 1] or RTFSRLAM [SEQ ID NO. 2] (CDR1), GISRSGDGTHYAYSV [SEQ ID NO. 3] (CDR2), and AAARTAFYYYGNDYNY [SEQ ID NO. 4] (CDR3). Alternatively, the single domain antibody may comprise the sequence:

[SEQ ID NO. 5]
AIAIAVALAGFATVAQAQVKLEESGGGLVQAGGSLRLSCAASGRTSRR
YAMGWFRQAPGKEREFVAGISRSGDGTHYAYSVKGRFTISRDNAANT
VELQMNSLKPEDTAVYFCAAARTAFYYYGNDYNYWGQGTQVTVSS,

or a sequence substantially identical thereto. In another alternative, the sdAb may comprise the sequence:

[SEQ ID NO. 6]
AIAIAVALAGFATVAQAQVKLEESGGGSVQPGGSLRLSCAASGRTFSRL
AMGWFRQAPGKERELVAGISRSGDGTHYAYSVKGRFTISRDNAANTV
ELQMNSLKPEDTAVYFCAAARTAFYYYGNDYNYWGQGTQVTVSS,

or a sequence substantially identical thereto.

In another embodiment, the antibody may be IgG C225 (Gridelli et al, 2009, the contents of which are hereby incorporated by reference where permitted), or an antibody with a substantially identical sequence thereto. The antibody may also be an antibody fragment based on or obtained from IgG C225, retaining the binding specificity of IgG C225.

A sequence that is substantially identical to another sequence may comprise one or more conservative amino acid mutations. It is known in the art that one or more conservative amino acid mutations to a reference sequence may yield a mutant polypeptide with no substantial change in physiological, chemical, or functional properties compared to the reference sequence; in such a case, the reference and mutant sequences would be considered “substantially identical” polypeptides. Conservative amino acid mutation may include addition, deletion, or substitution of an amino acid; a conservative amino acid substitution is defined herein as the substitution of an amino acid residue for another amino acid residue with similar chemical properties (e.g. size, charge, or polarity).

In a non-limiting example, a conservative mutation may be an amino acid substitution. Such a conservative amino acid substitution may substitute a basic, neutral, hydrophobic, or acidic amino acid for another of the same group. By the term “basic amino acid” it is meant hydrophilic amino acids having a side chain pK value of greater than 7, which are typically positively charged at physiological pH. Basic amino acids include histidine (His or H), arginine (Arg or R), and lysine (Lys or K). By the term “neutral amino acid” (also “polar amino acid”), it is meant hydrophilic amino acids having a side chain that is uncharged at physiological pH, but which has at least one bond in which the pair of electrons shared in common by two atoms is held more closely by one of the atoms. Polar amino acids include serine (Ser or S), threonine (Thr or T), cysteine (Cys or C), tyrosine (Tyr or Y), asparagine (Asn or N), and glutamine (Gln or Q). The term “hydrophobic amino acid” (also “non-polar amino acid”) is meant to include amino acids exhibiting a hydrophobicity of greater than zero according to the normalized consensus hydrophobicity scale of Eisenberg (1984). Hydrophobic amino acids include proline (Pro or P), isoleucine (Ile or I), phenylalanine (Phe or F), valine (Val or V), leucine (Leu or L), tryptophan (Trp or W), methionine (Met or M), alanine (Ala or A), and glycine (Gly or G). “Acidic amino acid” refers to hydrophilic amino acids having a side chain pK value of less than 7, which are typically negatively charged at physiological pH. Acidic amino acids include glutamate (Glu or E), and aspartate (Asp or D).

Sequence identity is used to evaluate the similarity of two sequences; it is determined by calculating the percent of residues that are the same when the two sequences are aligned for maximum correspondence between residue positions. Any known method may be used to calculate sequence identity; for example, computer software is available to calculate sequence identity. Without wishing to be limiting, sequence identity can be calculated by software such as NCBI BLAST2 service maintained by the Swiss Institute of Bioinformatics (and as found at http://ca.expasy.org/tools/blast/), BLAST-P, Blast-N, or FASTA-N, or any other appropriate software that is known in the art.

The substantially identical sequences of the present invention may be at least 75% identical; in another example, the substantially identical sequences may be at least 70, 75, 80, 85, 90, 95, or 100% identical at the amino acid level to sequences described herein. Importantly, the substantially identical sequences retain the activity and specificity of the reference sequence.

As would be understood by a person of skill in the art, other antibodies may be used in the nanoconjugate of the invention. The antibody may be chosen in accordance with the desired target for imaging.

The antibody may be bioconjugated (also referred to herein as “conjugated”, “linked” or “coupled”) to the ULV, using any suitable method known in the art. For example, and without wishing to be limiting, the single domain antibody may be linked to the PEG-DPSE moiety prior to formation of the ULV, through a functional group such as a carboxylate, a sulfonate, a phosphate, an amine, and any combination thereof.

If a PEG molecule is used, conjugation of antibody to the PEG molecule may be accomplished using methods well known in the art (see for example Hermanson, 1996). Antibodies and single domain antibodies in particular, have several exposed lysine (primary amine) residues, and thus one method of covalently anchoring the antibody to the carboxylic acid-modified nanoparticle surface is through bioconjugation chemistry. Suitable coupling reagents include 1-ethyl-3-(3-dimethylaminopropyl) carbodiimide hydrochloride (EDC) which is often used in combination with N-hydroxysuccinimide (NHS). For example, the antibody as described above may have, or may be engineered to have, one or more lysine residues opposite or away from its antigen binding site, which is used in covalent conjugation to the nanoparticle surface. In one embodiment, the number of antibodies conjugated to the surface of the nanoparticle is controllable and controlled.

Alternatively, the antibody may be conjugated to the PEG molecule through an amino acid with a carboxylic acid (i.e., Glu or Asp) on the antibody and primary amines on the PEG, or through binding of the PEG (detecting entity) to a molecule that has binding activity towards the antibody and is already attached to the PEG molecule. For example, this molecule could be an antibody which binds to the antibody or to tags (C-Myc tag, His6 tag) on the antibody such as anti-C-Myc or anti-His6 antibodies, or through binding of a biotinylated antibody to a biotin binder on the surface of nanoparticles. Biotin binders are well known and may include streptavidin, neutravidin, avidin, or extravidin. The antibody could also be coupled to the nanoparticle by means of nickel-nitrilotriacetic acid chelation to a His6-tag.

In another alternative, antibodies can also be engineered to have cysteines opposite their antigen binding sites. Conjugation via a maleimide cross-linking reaction allows the directional display of antibodies where all antibodies are optimally positioned to bind to their antigens. Amine-terminated PEG molecule is activated with maleimide in DMF followed by an incubation of cysteine-terminated single domain antibody to achieve covalent binding through the formation of sulfide bond formation.

The number of antibody molecules conjugated to the surface of the ULV may vary, based on various factors, such as the size of the ULV. The conjugate of the present invention may comprise at least 1 to 100 antibody molecules conjugated to the surface of the ULV; for example, the conjugate may carry at least 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, or 100 antibody moieties linked to the ULV. As a person of skill in the art would recognize, it may be possible to conjugate more antibody molecules to the surface of the nanoparticle, depending on particle size, antibody size and characteristics, and on immobilization efficiency.

It is to be noted that each of the antibody molecules linked to the nanoconjugate may be the same, or may differ from one another. Thus, the ULV may be conjugated to more than one antibody to detect multiple target molecules simultaneously. The ULV may be conjugated to different antibodies that recognize different parts (epitopes) on the same pathogen, for example, but not limited to different epitopes on the same toxin or different epitopes on the same bacterial cell surface molecules or different epitopes on different cell surface molecules of the same bacteria.

As will be recognized by those skilled in the art, the diameter of the nanoconjugate may vary depending on the lipid composition used and the type of antibody conjugated to the ULV surface. Without wishing to be limiting in any manner, the overall size of the nanoconjugate of the present invention may be between about 20 and 200 nm in diameter. For example, and without wishing to be limiting, the nanoconjugate may have a diameter of about 20, 30, 40, 50, 60, 70, 80, 90, 100, 110, 120, 130, 140, 150, 160, 170, 180, 190, or 200 nm, or any value therebetween. In a specific, non-limiting example, the nanoconjugate diameter may be about 40 to about 100 nm. Nanoconjugates of the present invention comprising larger antibodies (such as IgG) may have larger diameters.

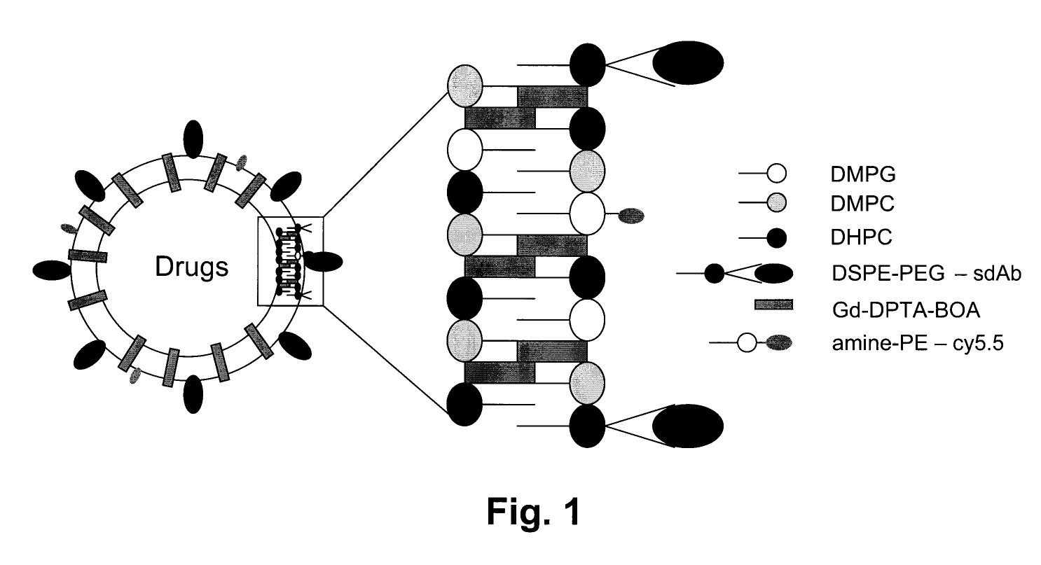

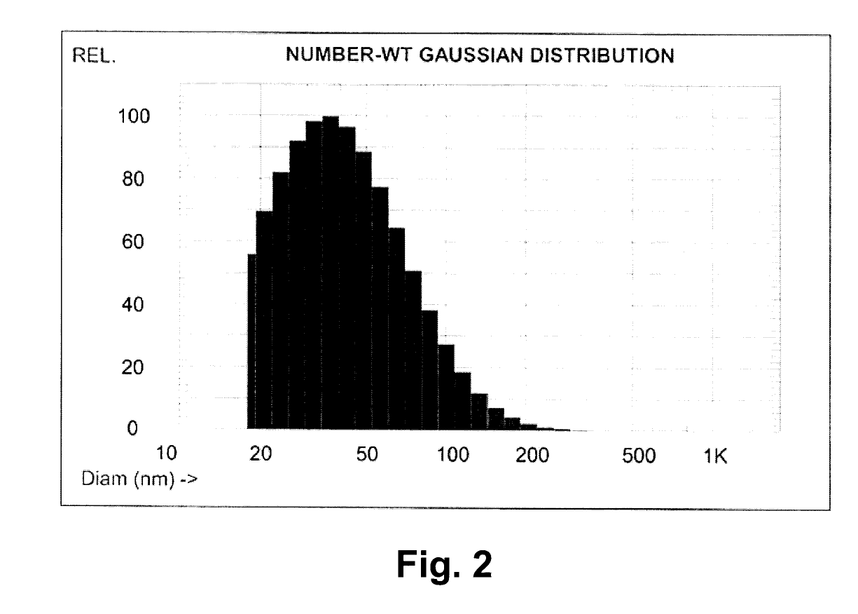

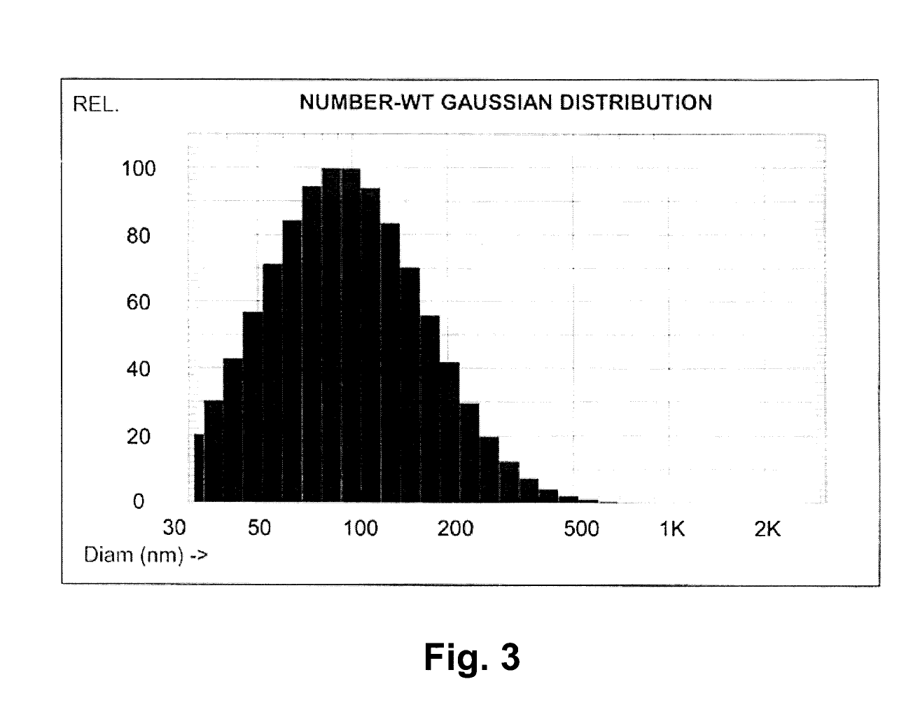

An exemplary embodiment of the nanoconstruct of the present invention comprising an ULV functionalized with sdAb and an optical imaging agent molecule is shown in the schematic of FIG. 1.

Formulations and compositions comprising the nanoconstruct of the present invention are also provided. In addition to the nanoconstruct of the present invention, such formulations or compositions may include pharmaceutically acceptable excipients or diluents, buffers, and/or water. The formulations may be powder, suspensions, or any other suitable pharmaceutical formulation.

The present invention further provides a method of forming unilamellar vesicles (ULV) incorporating at least one contrast agent, the method comprising:
(a) mixing dimyristoyl phosphatidylcholine (DMPC); dihexanoyl phosphatidylcholine (DHPC); dimyristoyl phosphatidylglycerol (DMPG); distearoyl phosphoethanolamine-[maleimide(polyethylene glycol)-2000] (DSPE-PEG-maleimide) and gadolinium-diethylene-triamine-pentaacetic acid bis-oleate (Gd-DTPA-BOA); and
(b) allowing the spontaneous formation of ULV.

In the method as described above, an antibody may be bioconjugated to DSPE-PEG-maleimide prior to step (a), thus incorporating the antibody into the nanoconjugate.

The present invention also provides a method for in vivo imaging of cells or tissues in a mammal, the method comprising the steps of:
(a) administering to the mammal a composition comprising a nanoconjugate as described herein, wherein the antibody is specific for a selected receptor;
(b) waiting a time sufficient to allow the antibody to bind to the selected receptor; and
(c) imaging the cells or tissues with a non-invasive imaging technique whose resolution is enhanced by the presence of the particles on or within the cells.

The imaging technique used may be selected from the group consisting of magnetic resonance imaging, magnetic spectroscopy, X-ray, positron emission tomography, optical imaging, computed tomography, and ultrasonic imaging. The method as described may allows for imaging of one or more tumors, metastases, vascularized malignant cell clusters, or individual malignant cells selected from the group consisting of brain cancer, colon cancer, breast cancer, prostate cancer, lung cancer, pancreatic cancer, endometrial cancer, oral cancer, liver cancer, and renal cancer or any other cancer.

In the method as described above, the selected receptor may be is specifically expressed by tumor endothelial cells. The selected receptor may be IGFBP7 or EGFR, and the antibody may be as described above.

Also provided is a method for detecting glioblastoma in a patient, comprising:
(a) contacting a tissue of interest with a nanoconjugate as described herein, wherein the antibody is specific for the IGFBP7 or EGFR, and may comprise the specific antibodies described above; and
(b) measuring the level of binding of the nanoconjugate, wherein an elevated level of binding, relative to normal tissue, is indicative that the tissue is neoplastic.

In yet another aspect, the present invention provides a method for detecting a tissue expressing IGFBP7, comprising:
(a) contacting a tissue of interest with a nanoconjugate as described herein, wherein the antibody is specific for the IGFBP7, and may comprise the specific antibodies described above; and
(b) measuring the level of binding of the nanoconjugate, wherein an elevated level of binding, relative to normal tissue is indicative of the presence of a tumor expressing IGFBP7.

In yet another aspect, the present invention provides a method for detecting a tissue expressing EGFR, comprising:
(a) contacting a tissue of interest with a nanoconjugate as described herein, wherein the antibody is specific for the EGFR, and may comprise the specific antibodies described above; and
(b) measuring the level of binding of the nanoconjugate, wherein an elevated level of binding, relative to normal tissue is indicative of the presence of a tumor expressing EGFR.

In the methods as just described, the step of measuring is performed by magnetic resonance imaging. The nanoconjugate used in the method as just described may further comprise a fluorescent imaging agent, and the step of detecting may be performed using fluorescence imaging.

The present invention also provides a method for determining the location of glioblastoma brain tumor cells in a patient pre-operatively, intra-operatively, and/or post-operatively, comprising the step of administering a composition comprising a nanoconjugate as described herein, wherein the antibody is specific for IGFBP7 or EGFR, and may comprise the specific antibodies described above, and a pharmaceutically acceptable carrier to the patient, wherein the composition is administered in an amount sufficient to image glioblastoma cells in vivo; and
(a) pre-operatively measuring the level of binding of nanoconstruct by magnetic resonance imaging to determine the location of glioblastoma cells, wherein an elevated level of binding, relative to normal tissue, is indicative of the presence of glioblastoma cells;
(b) intra-operatively measuring the level of binding of the nanoconstruct by fluorescence imaging to determine the location of residual glioblastoma cells, wherein an elevated level of binding, relative to normal tissue, is indicative of the presence of residual glioblastoma cells;
(c) post-operatively measuring the level of binding of the nanoconstruct by magnetic resonance imaging to determine the location of glioblastoma cells, wherein an elevated level of binding, relative to normal tissue, is indicative of the presence of tumor cells; or
(d) a combination of (a), (b) or (c) above.

A method for in vitro detection or quantification of biological or chemical molecule in a sample is also provided by the present invention. The method comprises the steps of:
(a) contacting the sample with a solution comprising a nanoconjugate as described herein, so as to form a complex between the molecule and the nanoconjugate; and
(b) detecting or quantifying said complex formed.

The step of detecting or quantifying may be performed by magnetic resonance imaging, fluorescence imaging, or a combination thereof.

In one embodiment, monoclonal antibodies against Epidermal Growth Factor (225 mAb) and single domain antibodies against IGFBP7 may be conjugated to the ULV nanoparticle, while retaining their full activity. The IGFBP7 sdAb-targeted bi-modal ULVs are selectively targeted to orthotopic brain tumors in nude mice, which may be demonstrated using optical in vivo imaging modality and in vivo MRI imaging. The C225 (anti-EGFR IgG)-targeted bi-modal ULVs are selectively targeted to xenograft tumors expressing EGFR in nude mice, which may also be demonstrated using optical in vivo imaging modality. The presence of C225 targeted ULVs in the xenograft tumors may be confirmed by fluorescence microscopy based detection of Cy5.5 in tumor sections. Excised xenograft tumors after injection of C225 (anti-EGFR IgG)-targeted bi-modal ULVs exhibit high Gd concentrations (measured by ICP-MS) The presence of C225 targeted ULVs in the xenograft tumors may also be confirmed by fluorescence microscopy based detection of Cy5.5 in tumor sections.

ULV nanoparticle formulations with high payload of Gadolinium-DTPA-BOA and near-infrared imaging contrast agent, Cy5.5, targeted using single domain antibody against IGFBP7 or monoclonal IgG antibody against EGFR have been synthesized and tested in xenograft and orthotopic brain tumor models in nude mice. The targeted ULVs of the present invention may be used in non-invasive (molecular) diagnosis/imaging (optical, MRI) of brain tumors and other tumors expressing IGFBP7 or EGFR and exhibiting a high-rate of angiogenesis (i.e., colon, breast). When a therapeutic agent is combined with the ULVs, either by encapsulation or surface incorporation, such tumors may be treated by application of the ULVs. Therapeutic agents may include pharmaceutical agents such as anti-cancer drugs or biological agents such as immunotherapeutic agents.

### EXAMPLES

The following examples are intended to exemplify specific embodiments of the invention, and not to limit the claimed invention in any manner.

### Example 1

### Unilamellar Vesicle Production and their Loading with Gadolinium

Dimyristoyl phosphatidylcholine (DMPC), dimyristoyl phosphatidylglycerol (DMPG), dihexanoyl phosphatidylcholine (DHPC) and distearoyl phosphoethanolamine-N-[Maleimide(Polyethylene Glycol)2000] (DSPE-PEG2000-Maleimide) were purchased from Avanti Polar Lipids (Alabaster Ala.). The Gd-DTPA-BOA was custom synthesized. All chemicals were used without further purification.

The structures of two lipid mixtures (high-concentration Gd mixture (HC-Gd) and low-concentration Gd mixtures (LC-Gd)) with the following molar ratios: DMPC/DMPG/DHPC/DSPE-PEG2000-Maleimide/Gd-DTPA-BOA=100:1:47:10:40 and 100:1:78:16:129 for LC-Gd and HC-Gd, respectively, were examined. The molar ratios were selected to yield 20 and 40 mol % of Gd-DTPA-BOA to total lipid mixture while keeping a constant long-to-short chain lipid ratio of 3.2, DMPG/DMPC ratio of 0.01 as previously published (Nieh et al, 2004, 2005) and a constant DSPE-PEG2000-Maleimide/total lipid of 0.05. Specifically, the lipid mixtures comprised:
HC-Gd: Gd-DTPA-BOA=40 mol. %; DMPC=30.6 mol. %; DHPC=23.9 mol. %; DMPG=0.4 mol. %; DSPE-PEG-maleimide=5 mol. %; dode-PE=0.1 mol. %.
LC-Gd: Gd-DTPA-BOA=20 mol. %; DMPC=50.5 mol. %; DHPC=23.9 mol. %; DMPG=0.5 mol. %; DSPE-PEG-maleimide=5 mol. %; dode-PE=0.1 mol. %.

Both LC-Gd and HC-Gd mixtures were first dissolved in chloroform (>99.9% from Aldrich) at corresponding molar ratios and dried by continuously flowing N₂ gas followed by evacuation with vacuum for 24 hours. The dried samples were then completely re-dispersed in D₂O to form 10 wt % solutions by temperature cycling and vortex between 40 and 50° C. (4˜5 cycles). The 10 wt % solutions are liquid-like at low temperature (the low-temperature viscosity of HC-Gd is higher than that of LC-Gd) but gel-like at high temperature, illustrating the same phenomenon as previously reported in the phospholipid systems of spontaneously forming vesicles (Nieh et al, 2004, 2005). All the samples were then diluted with cold D₂O at 4° C. into total lipid concentrations, CLp of 5 wt %, 1 wt % and 0.2 wt %.

A third formulation (HC-Gd 20 nm) was developed subsequent to SANS analysis (see Example 2) with the goal of forming smaller ULV. The new formula retained the same amounts of Gd-DTPA-BOA found in the original formula, except that DMPC was completely replaced by the charged DMPG lipid. Specifically, the third formulation included 4 components, namely: 40 mol % of Gd-DTPA-BOA, 23.8 mol. % of DHPC, 31.2 mol. % of DMPG and 5.0 mol. % DSPE-PEG-amine. The total lipid concentrations of samples with this formula in D₂O were 0.5 wt. % and 1 wt. %. The formulations were prepared in a manner similar to that described above for HC-Gd and LC-Gd.

### Example 2

### Small Angle Neutron Scattering (SANS)

For small angle neutron scattering (SANS) study, the lipid mixtures were dissolved in deuterium oxide with a purity>99.9% (Chalk River Laboratories, ON, Canada) to enhance the neutron scattering contrast. All the SANS measurements are done at 50° C. where the lamellae are expected to form. SANS experiments were conducted at the 30 m NG7 SANS located at National Institute of Standards and Technology (NIST) Center for Neutron Researches (NCNR, Gaithersburg, Md., USA).

The wavelength (λ) of the incident neutrons was 6 Å and three sample-to-detector distances (1 m, 4 m and 15.3 m) were applied, covering a scattering vector (q) ranged from 0.003 Å⁻¹ to 0.3 Å⁻¹. Data were collected using a 2-D position-sensitive detector as a function of scattering angle (θ). The raw data were corrected for background (blocked beam), normalized with the monitored incident neutrons flux and sample transmission, and subtracted with equally treated empty cell scattering data. Finally, the reduced data were circularly averaged with respect to the beam center and put on an absolute intensity scale using the incident neutron flux and the sample thickness. The final scattering intensity, I, is presented as a function of scattering vector q, which is defined as

$$\frac{4 π}{λ} sin \frac{θ}{2} .$$

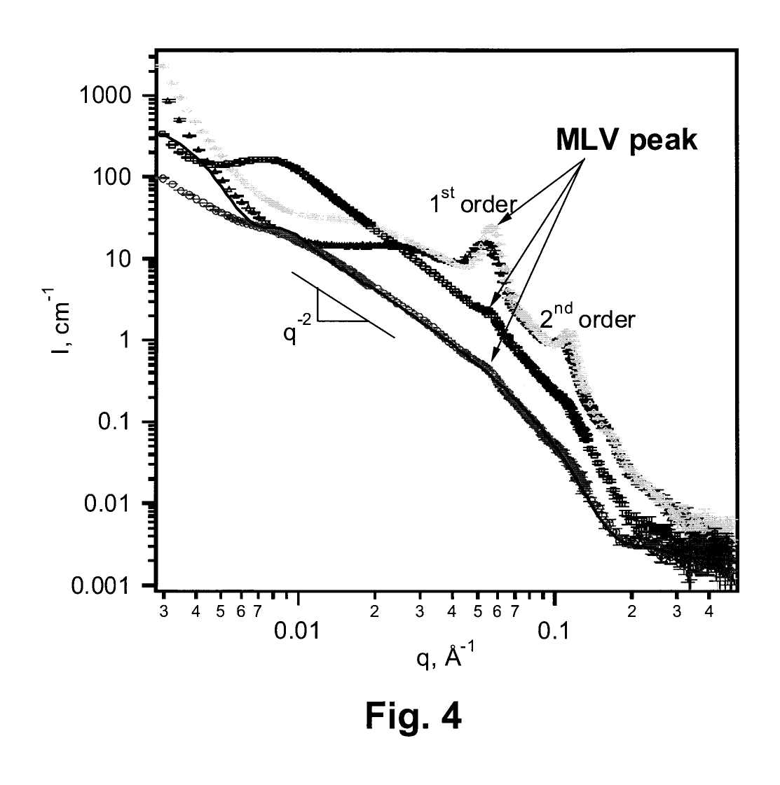

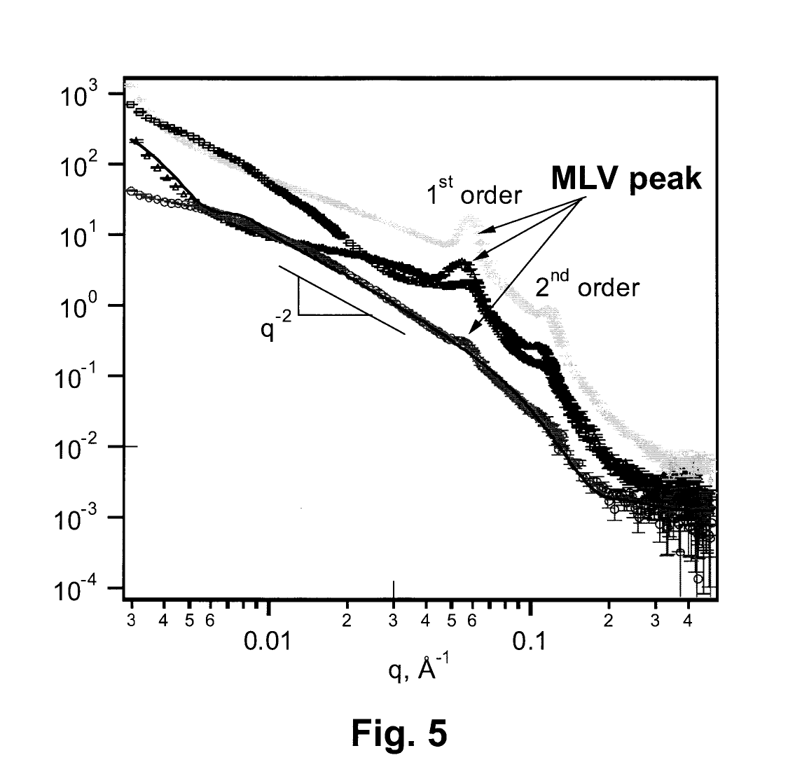

SANS data obtained from the original lipid mixtures, LC-Gd and HC-Gd, are shown in FIGS. 4 and 5, respectively. The q⁻² dependence at low- and mid-q indicated a bilayered structure, and the intensity oscillation at ˜q<0.01 Å⁻¹ corresponded to a characteristic length, mostly likely the size of the vesicles. At q˜0.06 Å⁻¹, a small, yet sharp peak was observed, indicating a well-defined distance originating from the interlamellar spacing of a multilamellar vesicle (MLV). In some cases, the second order peak (at q˜0.12 Å⁻¹) was also observed. Compared to the LC-Gd sample, this feature was more pronounced in the HC-Gd sample, indicating that Gd-DTPA-BOA may induce the formation of MLVs.

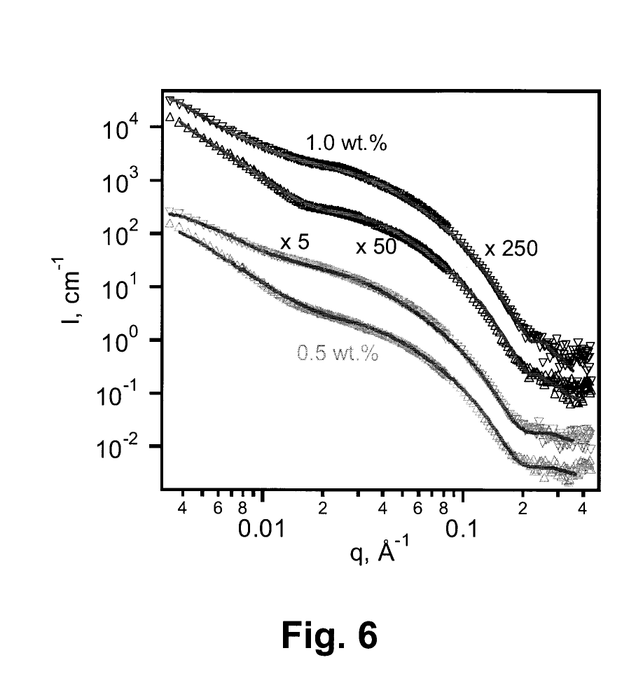

SANS experiments were similarly conducted on the third formulation at the 30 m NG3 SANS instrument located at the NIST (National Institute of Standards and Technology) Center for Neutron Research (NCNR, Gaithersburg, Md.). In order to study the stability of the resultant structure as a function of annealing time, SANS measurements were taken from both 0.5 and 1.0 wt. % samples incubated at 50° C. for either 18 hours or 3 days. SANS results indicated small differences between the two samples (FIG. 6). Importantly, the data did not exhibit any peaks associated with the previously observed MLV structure.

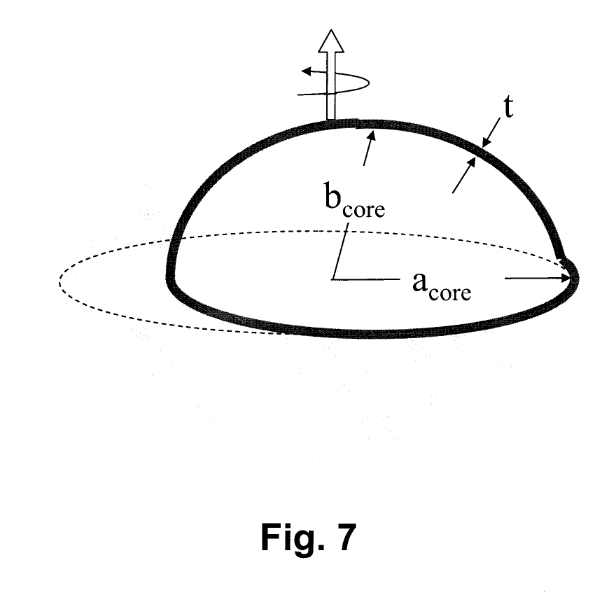

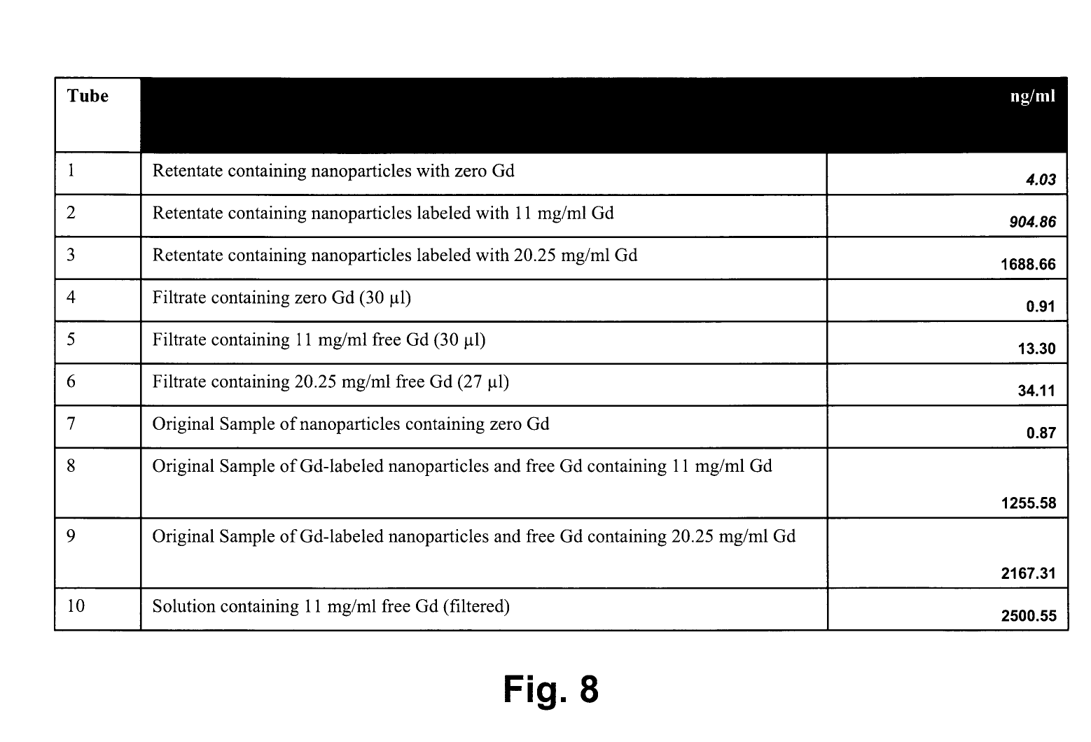

The SANS data were best fit using an ellipsoidal shell model (schematic shown in FIG. 7). This model includes four structural parameters: a) the long core axis (a_{core}); b) the short core axis (b_{core}); c) shell thickness (t) and d) the polydispersity of b_{core}. The best fit results are listed in Table 1. Although the SANS data showed significant differences in the low-q regime, the structural parameters obtained from the fits to the data did not differ dramatically, exception being the value of a_{core} which is greater in the 1.0 wt. % sample. The best fit results of 18 hour and 3 day (0.5 wt. % and 1 wt. % samples) data showed that they differed in contrast (Table 1). This difference in contrast between the two time period samples may be the result of the PEG chain rearranging at high temperature. However, the reason for the higher value of best fit result for scattering length density of the nanoparticle core, ρ_{core}, compared to that of D₂O, p_{D}_{₂}_{O}, is not understood. As would be understood by a person skilled in the art, the present model may be refined.

TABLE 1
Best-fit data from HC-Gd (20 nm)
at 50° C. for 18 hours and 3 days

| | 0.5 wt. % | | 1.0 wt. % | |
|---|---|---|---|---|
| | 18 hours | 3 days | 18 hours | 3 days |
| a_{core} (Å) | 625 | 570 | 1070 | 1230 |
| b_{core} (Å) | 65 | 59 | 60 | 64 |
| t (Å) | 38 | 38 | 39 | 38 |
| polydispersity | 0.48 | 0.5 | 0.49 | 0.46 |
| (ρ_{core} − ρₛₕₑₗₗ), | 6.6 × 10⁻⁶ | 6.2 × 10⁻⁶ | 6.7 × 10⁻⁶ | 6.0 × 10⁻⁶ |
| (Å⁻²) | | | | |
| (ρ_{D}_{₂}_{O} − ρₛₕₑₗₗ), | 4.1 × 10⁻⁶ | 4.6 × 10⁻⁶ | 4.2 × 10⁻⁶ | 4.7 × 10⁻⁶ |
| (Å⁻²) | | | | |

### Example 3

### Functionalization of Unilamellar Vesicles with Monoclonal Antibodies and Single Domain Antibodies

To enable targeting of the nanoconjugates to tumor-expressed antigens, ULVs were functionalized with antibodies attached to PEG-DSPE.

In one approach, ULVs were functionalized with a single-domain antibody against IGFBP7, newly discovered target selectively expressed in glioblastoma tumor vessels (see PCT Application No. PCT/CA2009/001460 and entitled Formulations of Targeting IGFBP7 for Diagnosis and Therapy of Cancer). Because the target is vascular, this formulation is suitable for imaging of intracranial tumors. Anti-IGFBP7 Single domain antibody was produced in-house. IGFBP7-sdAb was reconstituted in MES buffer (MES 0.1M, NaCl 0.5M, pH 6) using the aforementioned Amicon columns. To produce NHS-ester functionality on the sdAb, Sulfo-NHS and EDC were added to at 180- and 70-fold molar excess respectively and reacted for 30 min at room temperature. Subsequently, EDC was removed by centrifugation using Amicon columns.

In another approach, ULVs were functionalized with the monoclonal IgG C225 against Epidermal Growth Factor Receptor (EGFR) of glioblastoma cells (Gridelli et al, 2009). Because this target is expressed in glioblatoma cells, xenograft (flank) tumor model was used to provide proof of targeting. C225 antibody was reconstituted in MES buffer (MES 0.1M, NaCl 0.5M, pH 6) using the aforementioned Amicon columns. To produce NHS-ester functionality on the antibody, Sulfo-NHS and EDC were added to at 180- and 70-fold molar excess respectively and reacted for 30 min at room temperature. Subsequently, EDC is removed by centrifugation using Amicon columns.

### Example 4

### Intracranial and Xeongraft Models of U87MG deltaEGFRvIII Glioblastoma in Nude Mice

U87MG deltaEGFRvIII is a highly malignant glioblastoma cell line derived from a human brain tumor and has been engineered to overexpress both EGFR and the EGFRvIII mutant receptor (Dr W. K. Cavenee, Ludwig Institute for Cancer Research, San Diego, Calif., USA). Cells were maintained in DME medium containing 10% fetal bovine serum (FBS), penicillin/streptomycin and 200 μg/ml of G418. Cells were grown at 37° C. in a humidified atmosphere of 5% CO₂. Before cell implantation, cells were harvested by trypsinization in EDTA/trypsin, washed in phosphate-buffered saline (PBS), and centrifuged at 200 g three times and cell density was determined. Cells were kept on ice until injection. Animal procedures were performed according to a protocol approved by Institution Animal Care Committee. Nude CD-1 mice, obtained from Charles River Laboratories, Inc. (Cambridge, Mass.) at 4-6 weeks of age. The animals were housed in cages, in groups of 3 maintained on a 12-h light/dark schedule with a temperature of 22° C. and a relative humidity of 50±5%. Food and water was available ad libitum. Mice were injected subcutaneously in the left foreleg with 2×10⁶ U87MG glioblastoma cells suspended in 100 μL of phosphate-buffered saline (PBS). The tumor bearing mice were subjected to in vivo imaging studies when the tumors reached 0.4 cm in diameter (10 d after implant).

For intracerebral stereotactic implantation of U87MG cells, mice underwent isofluorane deep anesthesia and the scalp was swabbed with iodine and alcohol. The skin was incised and a 10 μl syringe was used to inoculate 5 μl of 5×10⁴ U87MG deltaEGFRvIII cell suspension into the corpus striatum in the right hemisphere (3.0 mm deep; 1 mm anterior and 2 mm lateral to the bregma). The skin was sutured with three knots, followed by application of tissue glue. The animals developed solid tumors for 10 days before experiment started.

### Example 5

### In Vivo Near-Infrared Fluorescence Imaging

The mice prepared in Example 4 were anesthetized with 1.5% isoflurane administered with a face mask. Single domain antibodies or conventional antibodies (each at 80 nmol/kg) bioconjugated to ULVs carrying 40% Gd-DTPA-BOA and labeled with the near-infrared fluorescent probe, Cy5.5, were administered via tail vein using a 0.5-ml insulin syringe with a 27-gauge fixed needle (vehicle, 0.9% saline; injection volume, 120 ul). Mice (n=5-10 per group) were imaged using small animal time-domain eXplore Optix pre-clinical imager MX2 (Advanced Research Technologies, QC) prior to and at different time intervals (4 h, 8 h and 24 h) after nanoparticles injections. In all imaging experiments, a 670-nm pulsed laser diode with a repetition frequency of 80 MHz and a time resolution of 12 ps was used for excitation. The fluorescence emission at 700 nm was collected by a highly sensitive time-correlated single photon counting system and detected through a fast photomultiplier tube offset by 3 mm for diffuse optical topography reconstruction.

Each animal was positioned prone on a plate that was then placed on a heated base (36° C.) in the imaging system. A two-dimensional scanning region (ROI) encompassing the whole body or the head was selected via a top-reviewing real-time digital camera. The optimal elevation of the animal was verified via a side viewing digital camera. The animal was then automatically moved into the imaging chamber where laser excitation beam controlled by galvomirrors was moved over the selected ROI. Laser power and counting time per pixel were optimized at 30 mW and 0.5 s, respectively and these values were maintained constant during the entire experiment. The raster scan interval of 1 mm was held constant during the acquisition of each frame; 1024 points were scanned for each ROI. The data were recorded as temporal point-spread functions (TPSF) and the images were reconstructed as fluorescence intensity, and fluorescence concentration maps. Following the last imaging session, mice were sacrificed by perfusion, organs were removed, placed into an imaging system and imaged ex vivo as described above.

eXplore Optix OptiView software program (Advanced Research Technologies, QC) was used to estimate fluorescence intensity; 3D reconstruction software by Advanced Research Technologies (Montreal, QC) was used for reconstruction of topography and optical sectioning.

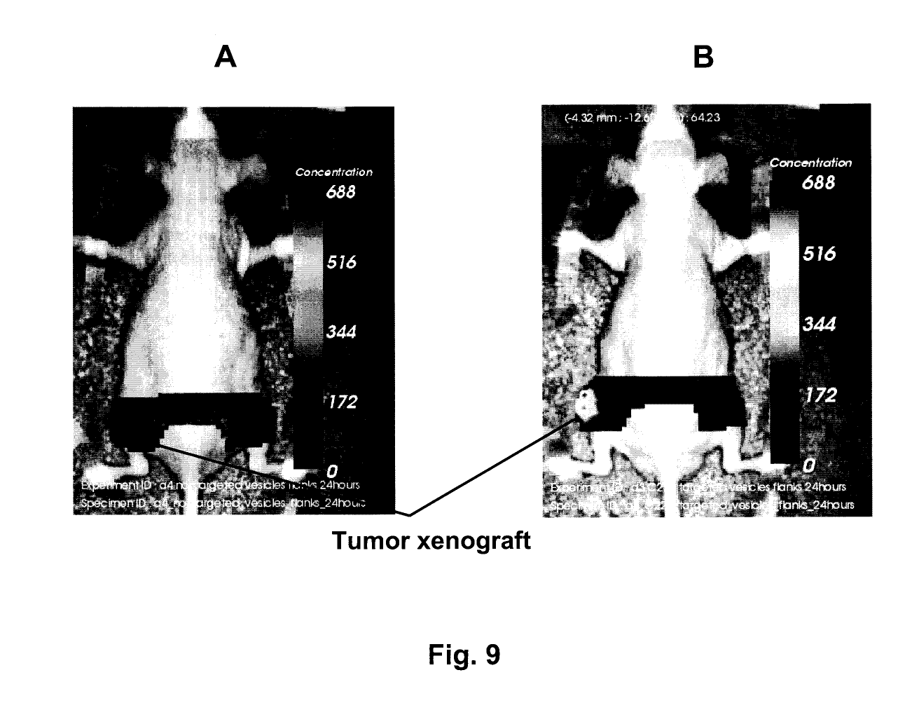

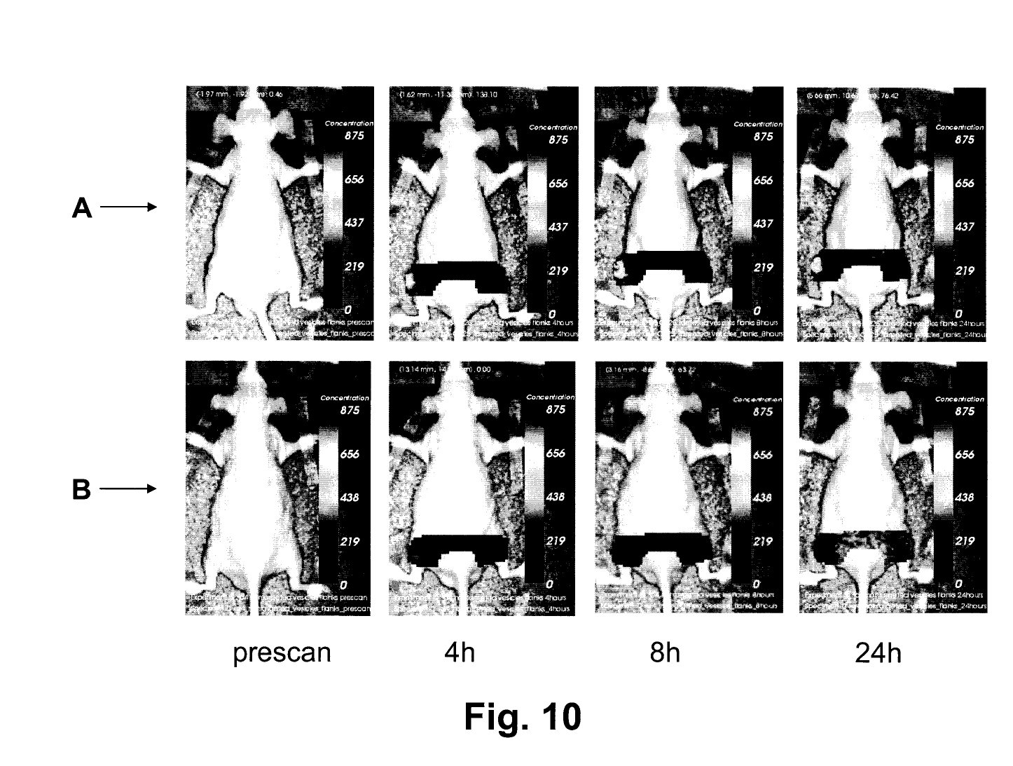

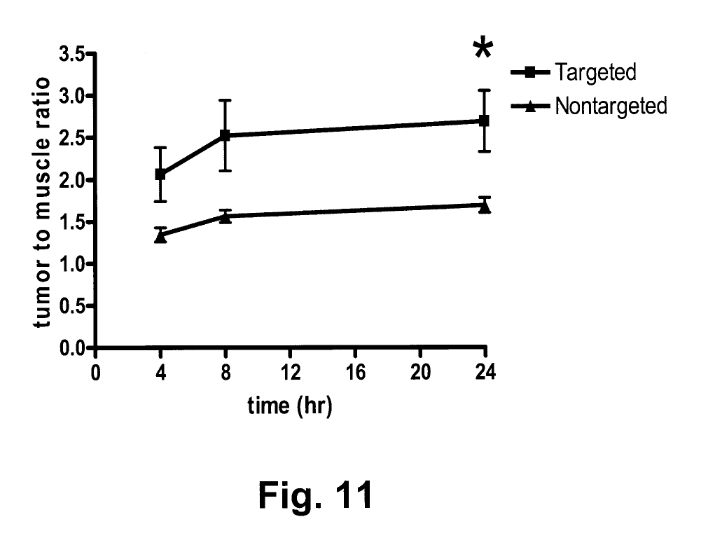

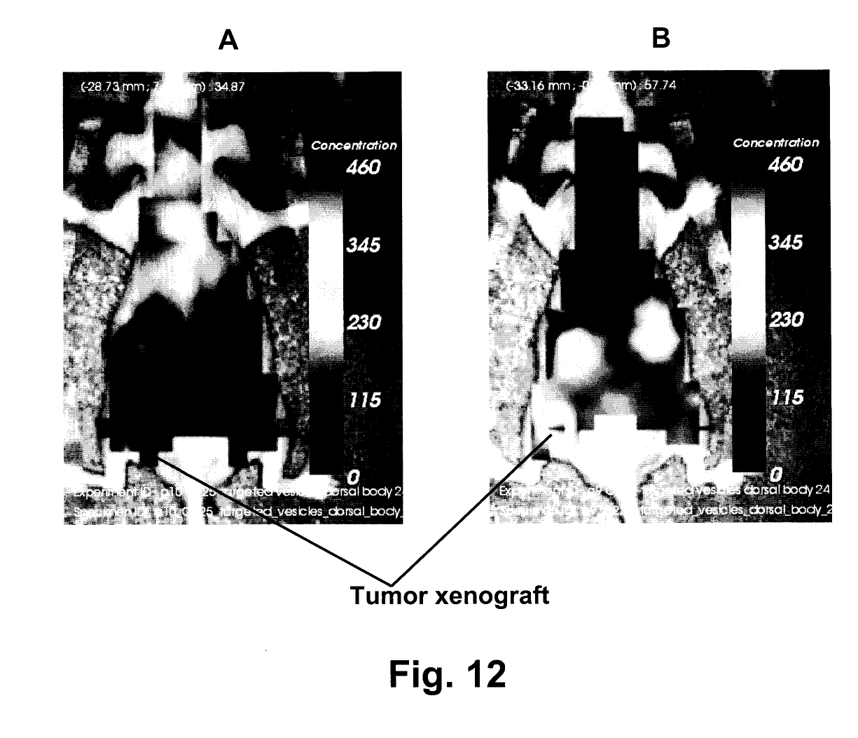

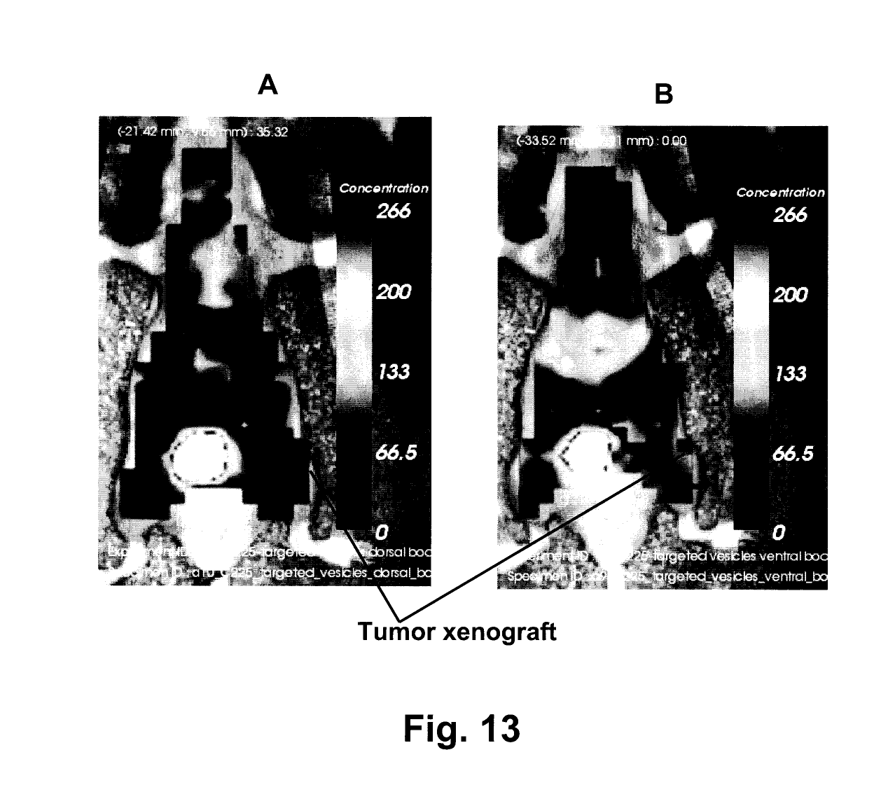

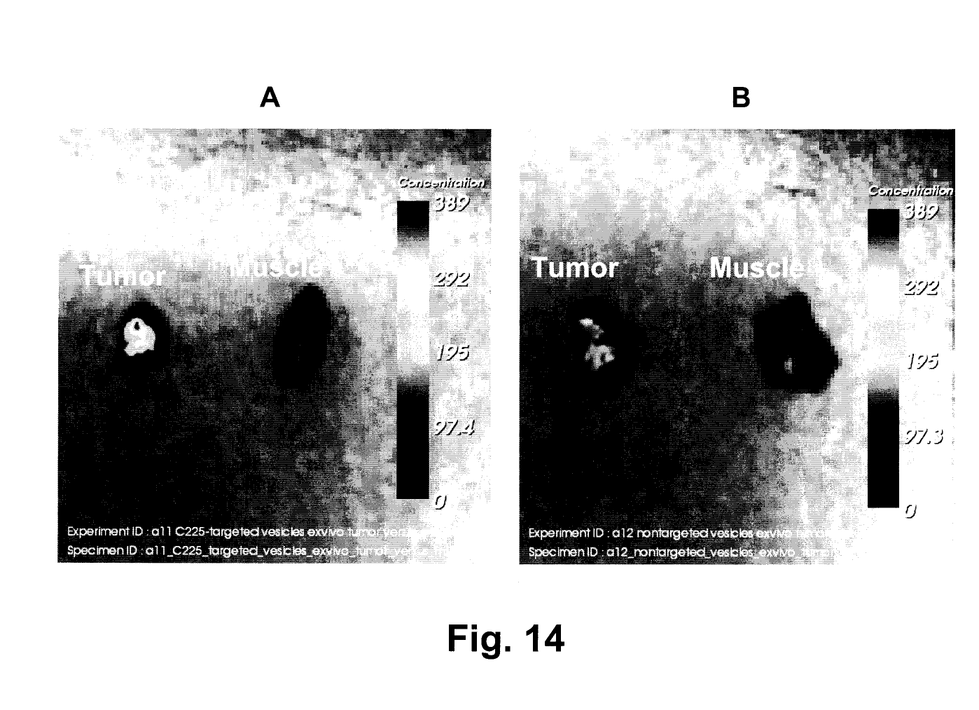

Results shown in FIGS. 9-14 demonstrate the targeting of C225 monoclonal antibody conjugated Gd-Cy5.5-ULV to EGFR-expressing subcutaneous xenograft tumors in nude mice. Cy5.5 fluorescence was only detected in C225 Ab-targeted ULV but not in non-targeted ULVs. Moreover, ex vivo tissue imaging of excised brain versus muscle showed fluorescence in brain tumor, but not in skeletal muscle tissue, only in animals injected with C225 targeted ULVs. In contrast, similar fluorescence was observed in excised brain tumor and skeletal muscle from animals injected with non-targeted ULVs.

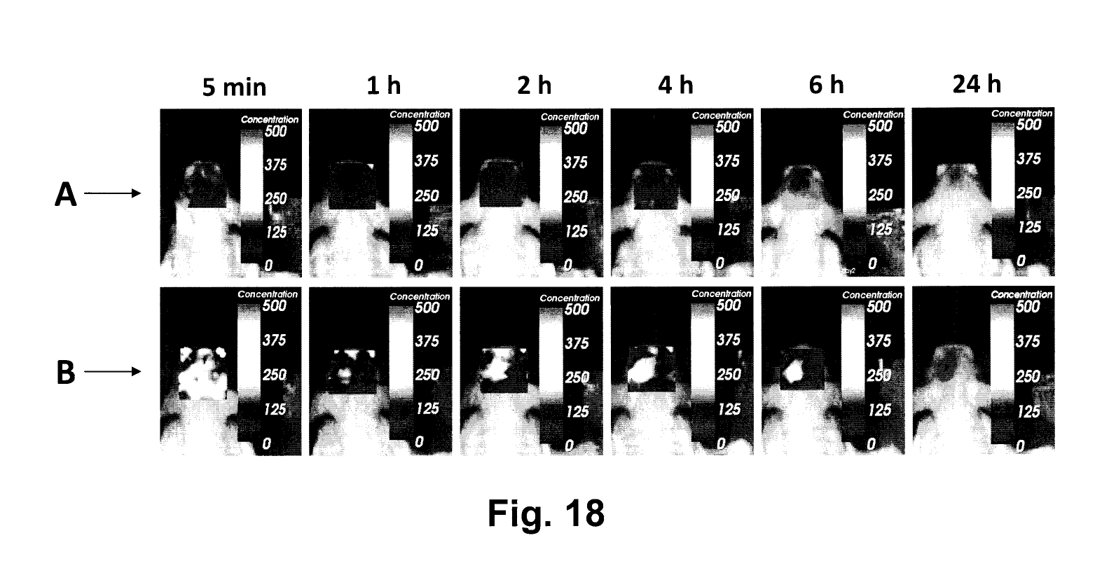

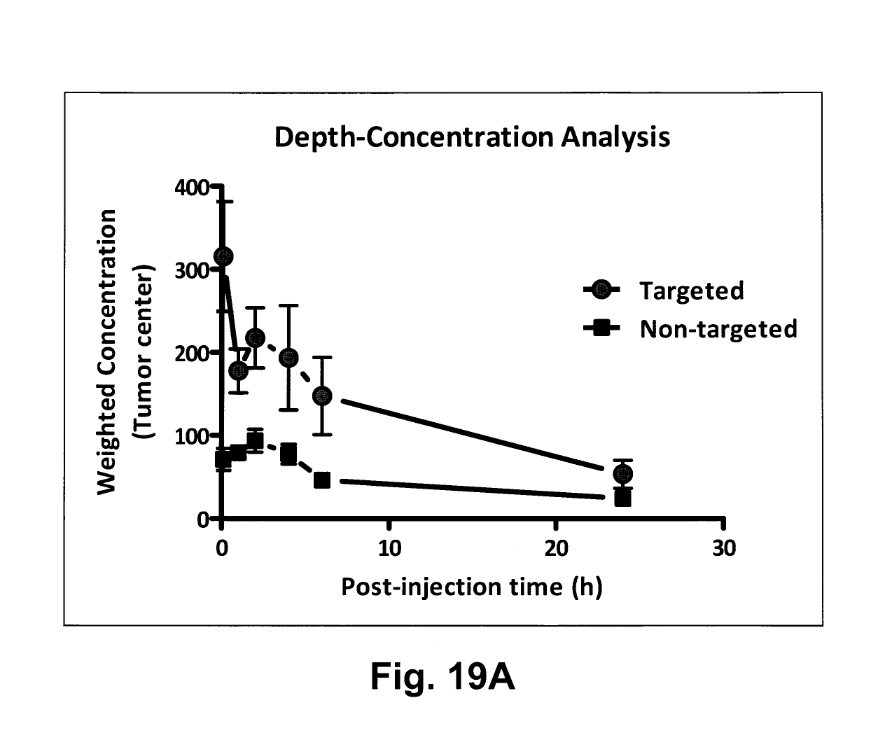

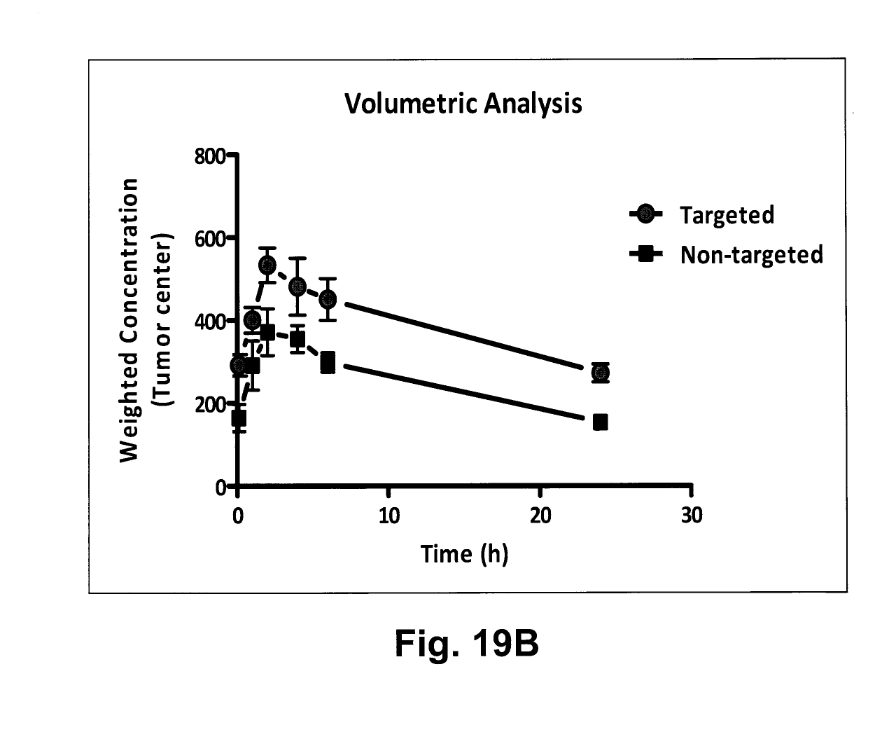

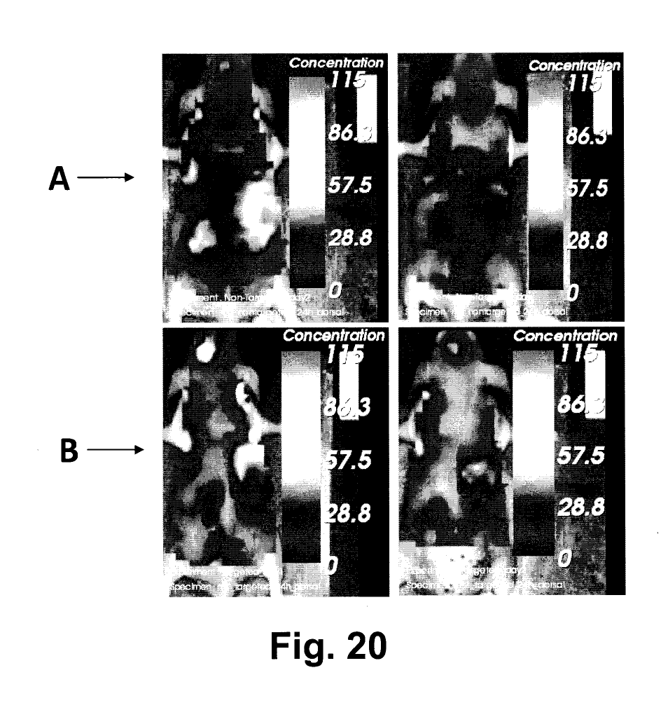

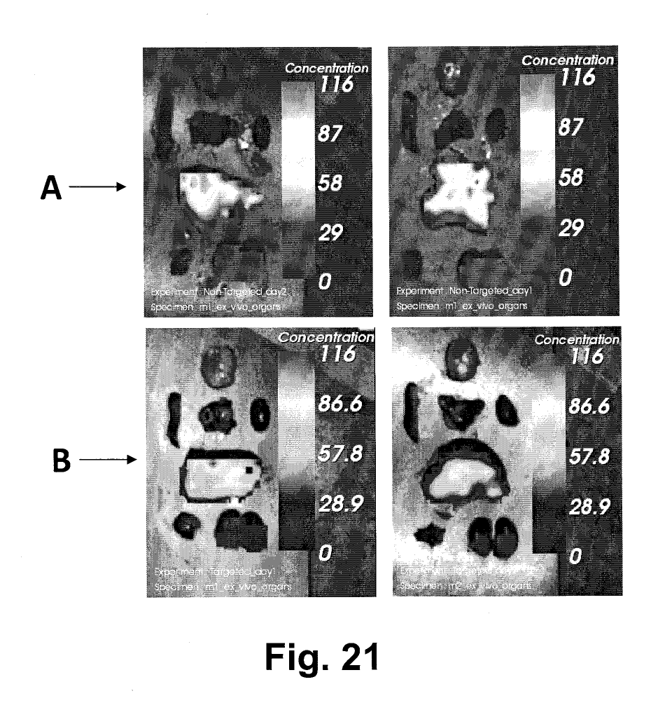

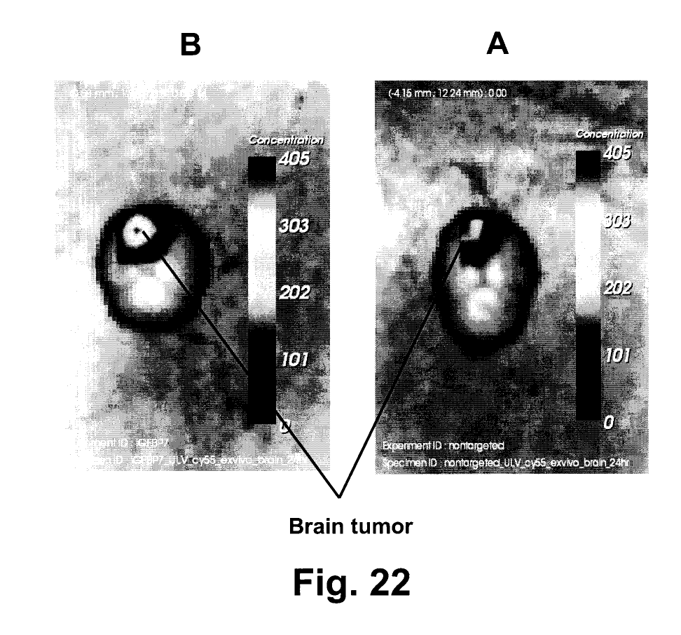

Results shown in FIGS. 18-22 demonstrate the targeting of anti-IGFBP7 single domain antibody conjugated Gd-Cy5.5-ULV to U87MG glioblastoma cells in orthotopic brain tumor model in nude mice. Cy5.5 fluorescence was only detected in IGFBP7 sAb targeted ULV but not in non-targeted ULVs. Moreover, ex vivo tissue imaging of excised brain showed fluorescence in brain tumor only in animals injected with IGFBP7-targeted ULVs, but not in excised brain tumor of animals injected with non-targeted ULVs

### Example 6

### Fluorescence Microscopy and Immunohistochemistry

After completion of the in vivo tumor imaging experiments of Example 5, animals were perfused with heparinized saline, organs and tumor were dissected and then frozen on dry ice and stored. Mouse tissues were embedded in Tissue-Tek freezing medium (Miles Laboratories, Elkhart, Ind.) and sectioned on a cryostat (Jung CM3000; Leica, Richmond Hill, ON, Canada) at 10 μm thickness, then mounted on Superfrost Plus microscope slides (Fisher Scientific, Nepean, ON, Canada). Slides were stored at −80° C. until immunohistochemical studies. Frozen mouse brain tumor sections were thawed for a few seconds then incubated in methanol for 10 min at room temperature. Slides were rinsed with 0.2 M PBS (pH 7.3), followed by incubation with 5% gpat serum in PBS for 1 hour with 0.1% triton-X 100 at room temperature. After blocking, slides were incubated with Anti-Epidermal Growth Factor Receptor (EGFR) antibody as a tumor biomarker and then visualized using goat anti-rabbit alexa 488 secondary antibody. Slides were then washed with PBS five times, dried of excess liquid and then coverslips were mounted using DAKO fluorescent mounting media. Coverslips were allowed to harden at 4° C. overnight and then visualized under fluorescent microscope.

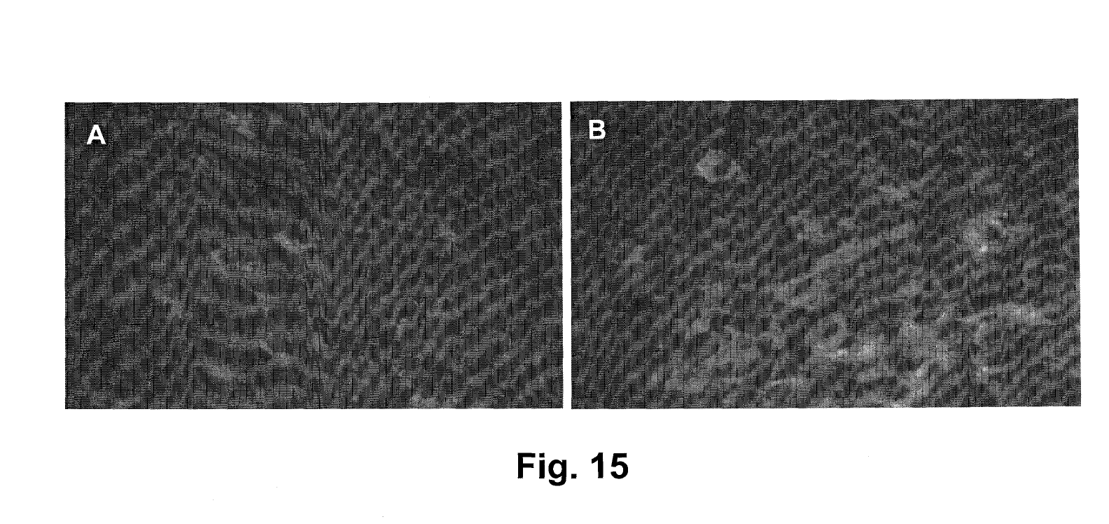

Results shown in FIG. 15 demonstrate the targeting of C225 Ab-Gd-ULV (Hc-Gd). Cy5.5 fluorescence was detected only in the tumor sections of animals injected with targeted ULV but not in animals injected with non-targeted ULVs.

### Example 7

### Sample Preparation for Determination of Total Gadolinium in Tissues Using Inductively Coupled Plasma Mass Spectrometry (ICP MS)

The ICP-MS instrument was an ELAN 6000 (PerkinElmer SCIEX, Thornhill, ON, Canada).

The digested samples were introduced into the ICP via a cross-flow nebulizer fitted in a Ryton spray chamber. Nitric acid was purified in-house prior to use by sub-boiling distillation of reagent-grade feedstock in a quartz still. High-purity de-ionized water (DIW) was obtained from a NanoPure mixed bed ion-exchange system fed with reverse osmosis domestic feed water (Barnstead/Thermolyne Corp, Iowa, USA).

Samples up to 50 mg of the freeze dried mice organs and tumors were digested in a PTFE vessel heated at 90° C. for 6 hours with 500 L nitric acid (69%) containing Rhodium (10 μg·L-1) as internal standard. The clear solution was diluted 15 times and analyzed with ICP MS. Concentrations of Gadolinium were determined by external calibration using values obtained after Rhodium normalisation.

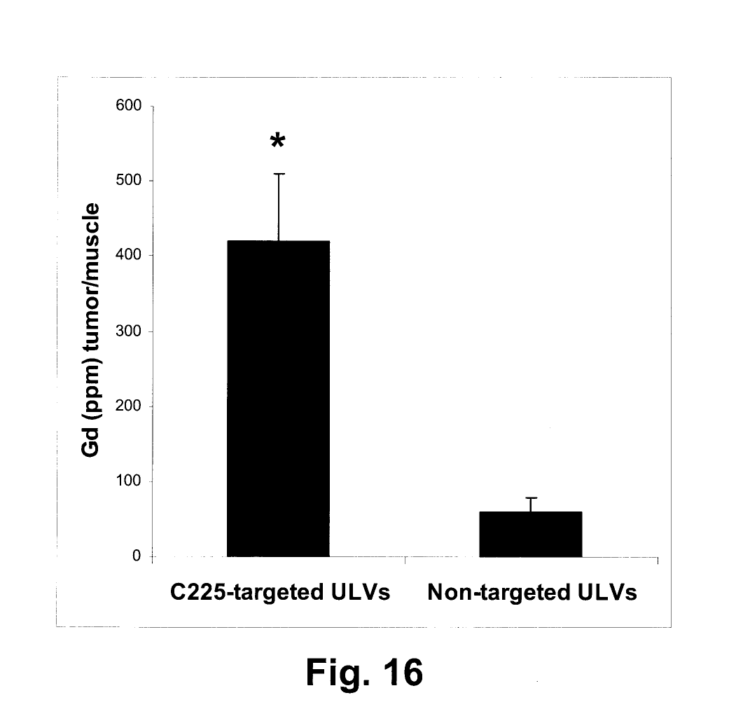

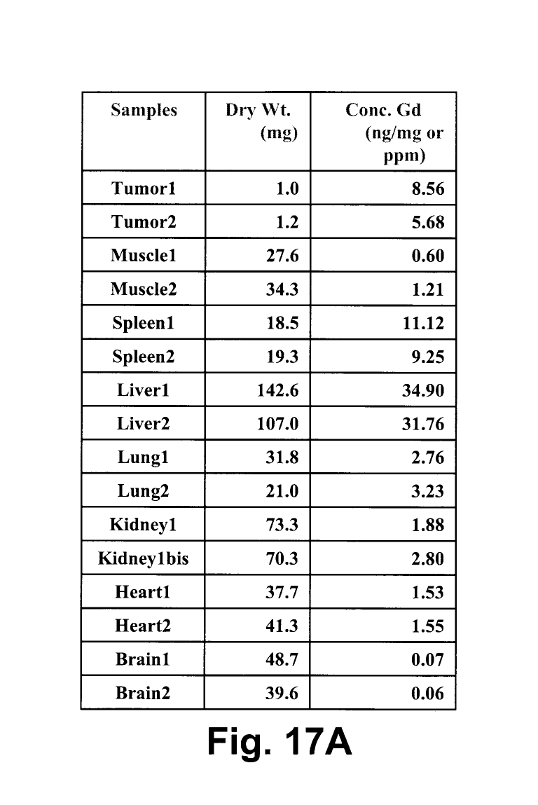

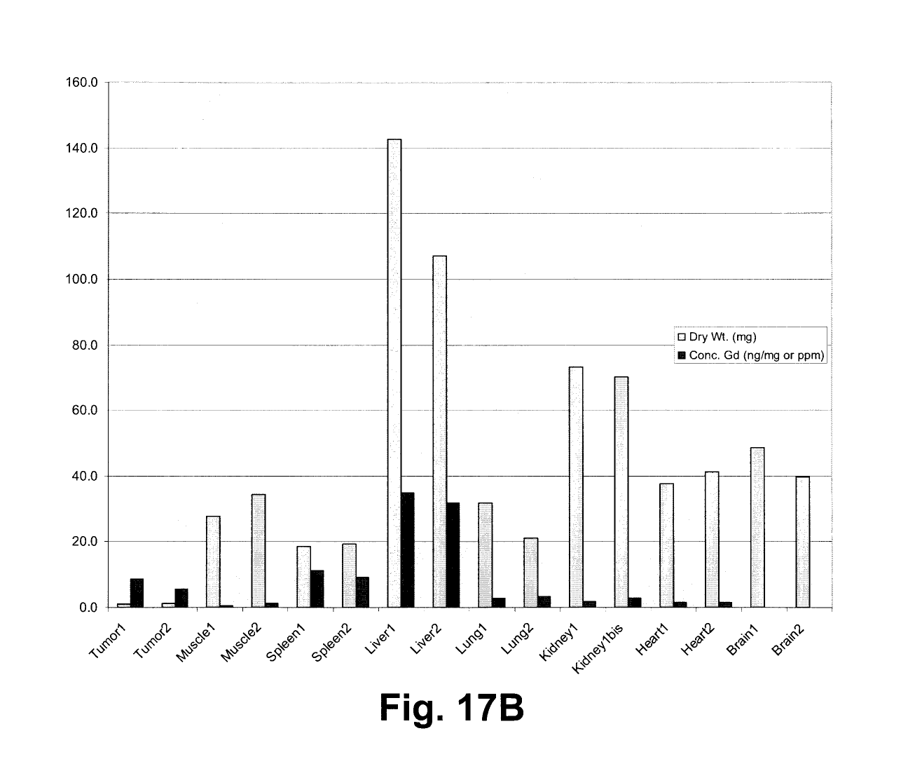

Results shown in FIG. 16-17 shows the quantitative measurements of Gadolinium in tissues from animals injected with C225-targeted or non-targeted Gd-Cy5.5-ULVs (HC-Gd). High number of Gd was measured in C225 monoclonal targeted ULV in tumor compared to low content of Gd in non-targeted ULV.

### Example 8

### MRI Measurement

The T1 relaxivity properties of samples containing the Gd loaded vesicles were compared to the T1 of samples containing unconjugated contrast agent or Gd-DTPA (Magnevist, Berlex, Canada). Solutions of 50 ug/ml or 200 ug/ml Gd concentration were prepared by diluting with distilled water the Gd-DTPA or Gd containing vesicles prepared with either 20 mol % or 40 mol % Gd-DTPA-BOA. These solutions (270 μl) were aliquoted into tubes which were embedded in agarose within a container. These samples were scanned using a quadrature coil and a Bruker Biospec Avance II MRI system with a 9.4 T magnet and Paravision 4 software. T1 maps of cross-sectional slices through the tubes were acquired using a RARE inversion-recovery sequence with variable repetition times. Three slices were acquired using a matrix of 128×128, field of view of 3 cm², TE=10 ms, flip angle of 180° and 10 different times of 135 ms, 375 ms, 630 ms, 950 ms, 1300 ms, 1750 ms, 2300 ms, 3100 ms, 4400 ms, 10000 ms. T1 for each sample was measured from the T1 maps calculated using the Paravision 4 software.

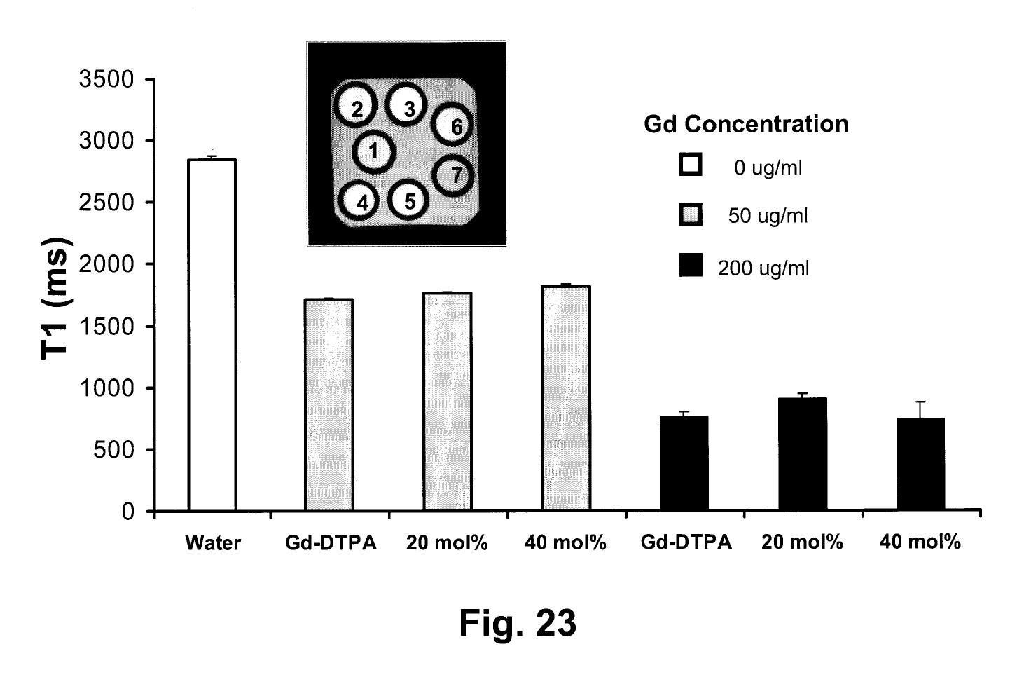

Results shown in FIG. 23 demonstrate the enhancement of T1 measurement by Gd-DTPA-BOA ULVs (HC-Gd 20 nm) in phantom using 9.4 T MRI.

### In Vivo Magnetic Resonance Imaging of IGFBP7 SdAb Targeted and Non-Targeted ULVs:

Tumor cells were implanted into CD-1 nude mice by injecting into the striatum 5 ul of cells slowly over 2-3 min. 7-8 days following cell implantation animals were scanned using standard T2 imaging (see below) to confirm successful tumor implantation. Nine to eleven days following injection, animals were anesthetized with isoflurane for contrast imaging using magnetic resonance (MR) imaging. First, the femoral vein was isolated and a catheter was inserted into the vein for contrast administration. The mouse was then moved into a cradle for positioning in the centre of a 9.4 T magnet equipped for MR imaging using a Bruker Biospec Avance II console. Animals were randomized to targeted or non-targeted contrast injection groups. In general prior to contrast injection T2, T1 weighted and T1 map scans were acquired. For the T2 map, a spin-echo sequence was acquired having 16 echoes with 10 msec echo spacing, a 2×2 cm² field of view, a 128×128 data matrix, a repetition time of 5000 ms, for each of 10 slices 1 mm thick. T₁ maps were acquired using a single shot echo planar sequence with a 2×2 cm² field of view, a 128×128 data matrix, a repetition time of 8.5 s, an echo time of 38 msec and 22 inversion time points every 400 ms for a 1 mm thick slice through the tumor. T₁ weighted images were acquired for 10 one mm thick slices with a RARE sequence using a 2×2 cm² field of view, a 128×128 data matrix, a repetition time of 750 ms, an echo time of 7.56 ms and 7 averages. T₂ and T1 maps were determined using local imaging software (Marevisi, National Research Council) and the Bruker Avance II software, respectively. After the pre-injection scans, either targeted or non-targeted contrast were injected intravenously (0.25 ml of HC-Gd 20 nm ULVs). In general, T1 maps were acquired repeatedly over the next 2 hours along with a T2 map and final T1 weighted scan. Effect of contrast was assessed using differences in intensity in the T1 weighted images by subtracting the pre-T1 weighted images from the final T1 weighted images. The effect of contrast on T1 values was also assessed by calculating T1 in tumor and contralateral unaffected brain prior to and following contrast injection.

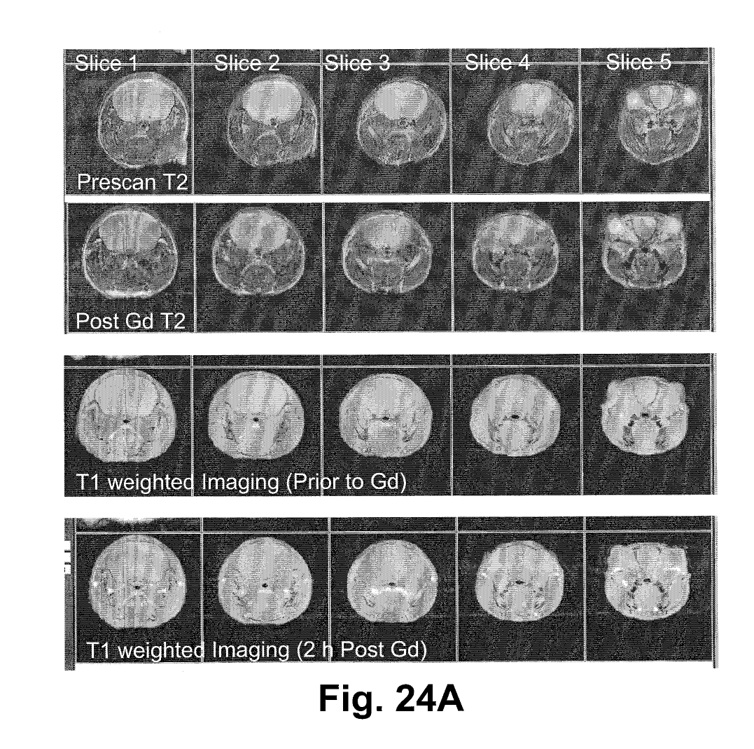

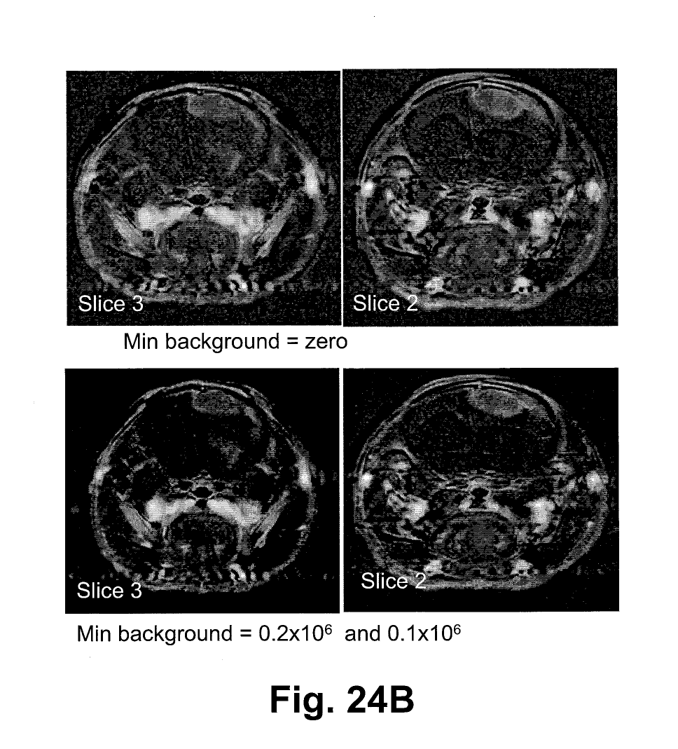

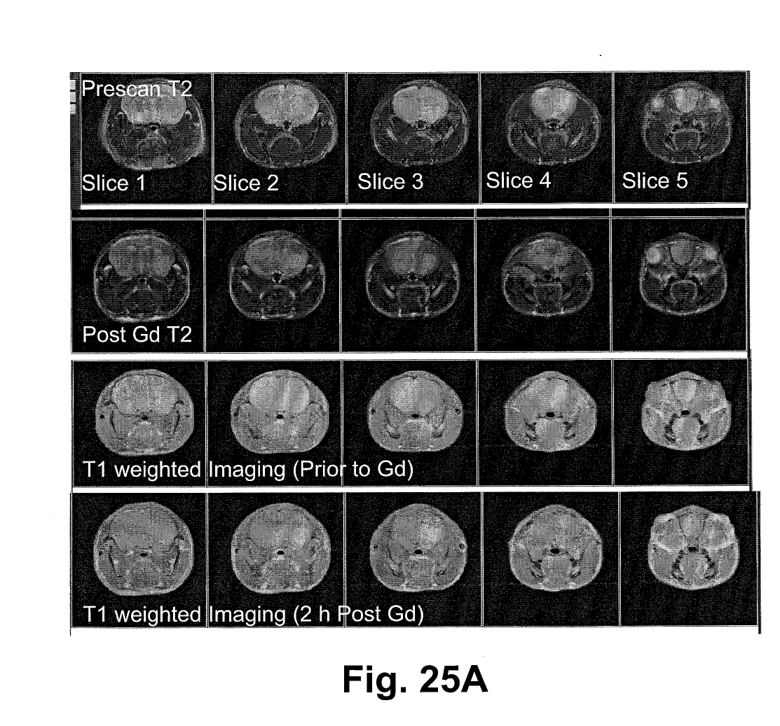

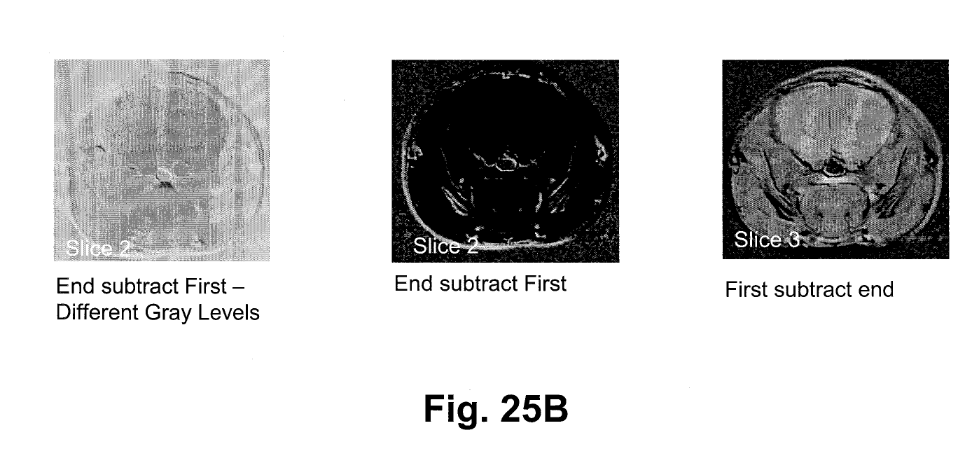

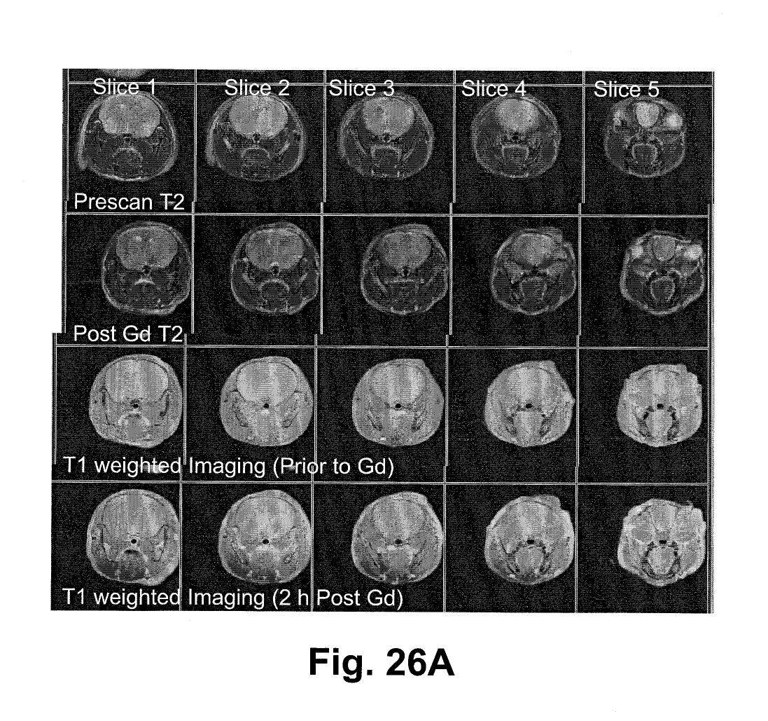

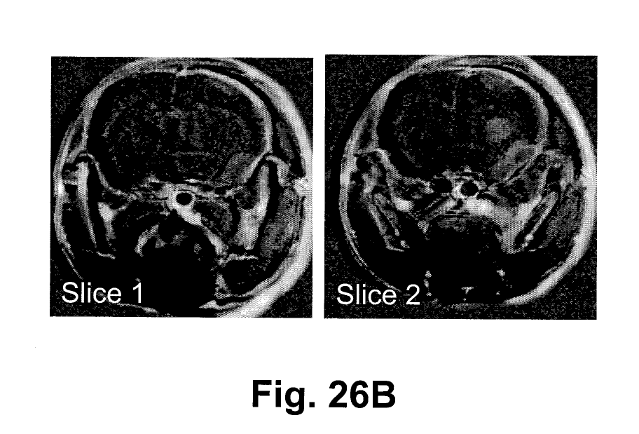

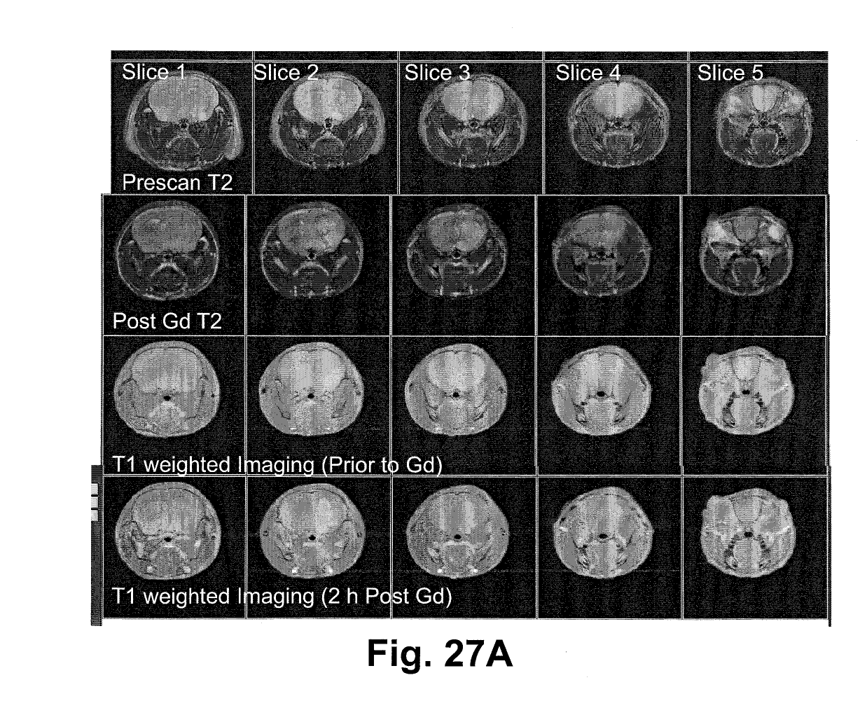

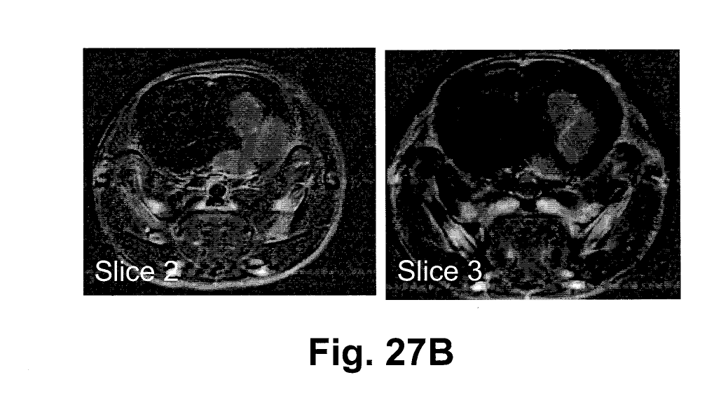

Results shown in FIG. 24-25 demonstrate the inability of non-targeted Gd-Cy5.5-ULVs vesicle (HC-Gd 20 nm) to image in vivo an implanted orthotopic brain tumor in nude mice using 9.4 T MRI. In contrast, FIG. 25-27 demonstrate the capability of anti-IGFBP7 sdAb targeted Gd-Cy5.5-ULVs vesicle (HC-Gd 20 nm) to image and visualized in vivo implanted orthotopic brain tumor in nude mice using 9.4 T MRI.

### Example 9

### Pharmacokinetic Analysis of Unilamellar Vesicles

Nanoparticles were injected via the tail vein in normal CD-1 mice. Blood samples of 25 μl volume were collected by creating a small nick in the tail vein followed by collection of blood in a heparanized tube. Blood samples were collected at multiple time points at 5 min, 30 min, 1 hr, 1.5 hr, 2 h, 4 h and 24 h. Samples were analyzed for labeled nanoparticles using a fluorescent plate reader with excitation 670 nm and emission 690 nm and compared to a standard curve of a range of known concentrations of the labeled nanoparticles diluted in whole blood. Pharmacokinetic parameters were calculated using the WinNonlin pharmacokinetic software package (Pharsight Corporation, CA). A one-compartment, IV-Bolus model was selected for pharmacokinetic modeling, as it best represented the actual data. This model is described by the following equation: C(t)=A exp (−αt) where C(t) represents the concentration of agent in serum. A represents the zero time intercept of the alpha phase, αis the disposition rate constants. Total clearance was determined from the equation Cl/F=D/AUC_{0-∞}.

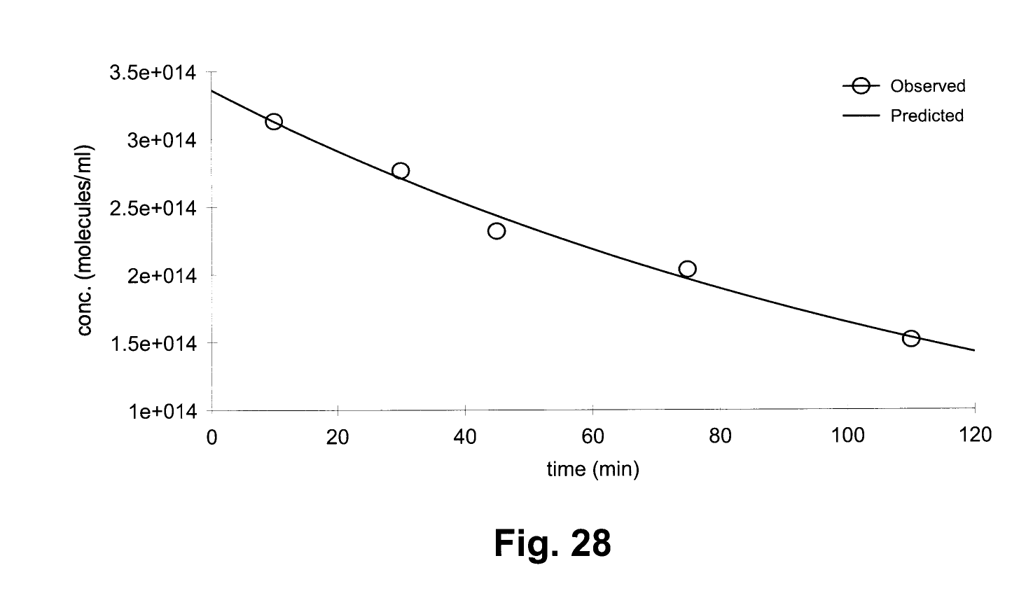

Results shown in FIG. 28 demonstrate the relatively fast clearance of unilamellar vesicles labelled with Cy5.5 (40 mol % Gd with 20 nm size) and injected intravenously in normal CD1 mice. Half life of the formulation was 97 minutes. This is necessary to obtain a high signal/background ratio and have an optimum image.

### REFERENCES

The following references are indicative of the level of skilled of one skilled in the art to which this invention relates, and are incorporated herein by reference as if reproduced in their entirety (where permitted).
Arbabi-Ghahroudi, M., To, R., Gaudette, N., Hirama, T., Ding, W., MacKenzie R., and Tanha, J. (2008). Aggregation-resistant VHs selected by in vitro evolution tend to have disulfide-bonded loops and acidic isoelectric points. PEDS. In press.
Arruebo M, Fernández-Pacheco R, Ibarra M R, Santamaría J. Magnetic nanoparticles for drug delivery Nano Today June 2007, Vol. 2, Issue-3, pp 22-32
Dooley, H., Flajnik, M. F., and Porter, A. J. (2003). Selection and characterization of naturally occurring single-domain (IgNAR) antibody fragments from immunized sharks by phage display. Mol. Immunol. 40: 25-33.
Gole A, Stone J W, Gemmill W R, zur Loye H C, Murphy C J. Iron oxide coated gold nanorods: synthesis, characterization, and magnetic manipulation. Langmuir. 2008, 17; 24:6232-7
Harmsen, M. M. and de Haard, H. J. (2007). Properties, production, and applications of camelid single-domain antibody fragments. Appl. Microbiol. Biotechnol. 77: 13-22.
Holliger, P. and Hudson, P. J. (2005). Engineered antibody fragments and the rise of single domains. Nat. Biotechnol. 23: 1126-1136.
Holt, L. J., Herring, C., Jespers, L. S., Woolven, B. P., and Tomlinson, I. M. (2003). Domain antibodies: proteins for therapy. Trends Biotechnol. 21: 484-490.
Hoogenboom, H. R. (2005). Selecting and screening recombinant antibody libraries. Nat. Biotechnol. 23: 1105-1116.
Huang, P., Tay, L., Tanha, J., Ryan, S, and Chau, L. Targeted detection of pathogenic bacteria, Staphylococcus aureus, with nano-aggregate embedded beads. In press.
Jespers, L., Schon, O., Famm, K., and Winter, G. (2004a). Aggregation-resistant domain antibodies selected on phage by heat denaturation. Nat. Biotechnol. 22: 1161-1165.
Jespers, L., Schon, O., James, L. C., Veprintsev, D., and Winter, G. (2004b). Crystal Structure of HEL4, a Soluble, Refoldable Human V(H) Single Domain with a Germ-line Scaffold. J. Mol. Biol. 337: 893-903.
Kell, K. Somaskandan, G. Stewart, M. G. Bergeron and B. Simard, Langmuir, 2008, 24, 3493-3502
LaConte L E, Nitin N, Zurkiya O, Caruntu D, O'Connor C J, Hu X, Bao G. Coating thickness of magnetic iron oxide nanoparticles affects R2 relaxivity. J Magn Reson Imaging. 2007; 26:1634-41
Liu, J. L., Anderson, G. P., Delehanty, J. B., Baumann, R., Hayhurst, A., and Goldman, E. R. (2007). Selection of cholera toxin specific IgNAR single-domain antibodies from a naive shark library. Mol. Immunol. 44: 1775-1783.
Lu A H, Salabas E L, Schuth F: Magnetic NPs: synthesis, protection, functionalization, and application. Angew. Chem. Int. Ed. Engl. 46(8), 1222-1244 (2007)
Lu, Y. Yin, B. T. Mayers and Y. Xia, Nano. Lett., 2002, 2, 183-186.D. Ma, T. Veres, L. Clime, F. Normandin, J. Guan, D. Kingston and B. Simard, J. Phys. Chem. C., 2007, 111, 1999-2007.
M.-P. Nieh, T. A. Harroun, V. A. Raghunathan, C. J. Glinka, J. Katsaras “Spontaneously formed monodispersed biomimetic unilamellar vesicles: the effect of charge, dilution and time” Biophys. J., 86, 2615-2629, (2004)
M.-P. Nieh, V. A. Raghunathan, S. R. Kline, T. A. Harroun, C.-Y. Huang, J. Pencer, J. Katsaras “Spontaneously formed unilamellar vesicles with path-dependent size distribution” Langmuir, 21, 6656-6661 (2005)
Magnetic resonance molecular imaging with nanoparticles. Lanza G M, Winter P M, Caruthers S D, Morawski A M, Schmieder A H, Crowder K C, Wickline S A. (2004) Magnetic resonance molecular imaging with nanoparticles. J Nucl Cardiol. 2004 November-December; 11(6):733-43.
Meincke M, Schlorf T, Kossel E, Jansen O, Glueer C C, Mentlein R. Iron oxide-loaded liposomes for MR imaging. Front Biosci. 2008, 1; 13:4002-8
Multimodality molecular imaging of tumor angiogenesis. Cai W, Chen X. (2008) Multimodality molecular imaging of tumor angiogenesis. J Nucl Med. 2008 June; 49 Suppl 2:113 S-28S.
Nuttall, S. D., Irving, R. A., and Hudson, P. J. (2000). Immunoglobulin VH domains and beyond: design and selection of single-domain binding and targeting reagents. Curr. Pharm. Biotechnol. 1: 253-263.
Nuttall, S. D., Krishnan, U. V., Hattarki, M., De Gori, R., Irving, R. A., and Hudson, P. J. (2001). Isolation of the new antigen receptor from wobbegong sharks, and use as a scaffold for the display of protein loop libraries. Mol. Immunol. 38: 313-326.
Revets, H., De Baetselier, P., and Muyldermans, S. (2005). Nanobodies as novel agents for cancer therapy. Expert. Opin. Biol. Ther. 5: 111-124.
Tanha, J., Dubuc, G., Hirama, T., Narang, S. A., and MacKenzie, C. R. (2002). Selection by phage display of llama conventional V(H) fragments with heavy chain antibody V(H)H properties. J. Immunol. Methods 263: 97-109.
Tanha, J., Nguyen, T. D., Ng, A., Ryan, S., Ni, F., and MacKenzie, R. (2006). Improving solubility and refolding efficiency of human V(H)s by a novel mutational approach. Protein Eng Des Sel 19: 503-509.
Tanha, J., Xu, P., Chen, Z. G., Ni, F., Kaplan, H., Narang, S. A., and MacKenzie, C. R. (2001). Optimal design features of camelized human single-domain antibody libraries. J. Biol. Chem. 276: 24774-24780.
Wang L, Luo J, Fan Q, Suzuki M, Suzuki I S, Engelhard M H, Lin Y, Kim N, Wang J Q, Zhong C J. Monodispersed core-shell Fe3O4@Au nanoparticles. J Phys Chem B. 2005; 109:21593-601
Wijaya A, Hamad-Schifferli K. High-density encapsulation of Fe3O4 nanoparticles in lipid vesicles. Langmuir. 2007; 23:9546-50
Woodcock J, Woosley R. The FDA critical path initiative and its influence on new drug development. Annu Rev Med. 2008; 59:1-12
PCT Application No. PCT/CA2009/001460 and entitled Formulations of Targeting IGFBP7 for Diagnosis and Therapy of Cancer

We claim:

1. A nanoconjugate comprising:
(a) a self-assembled unilamellar vesicle (ULV);
(b) at least one contrast agent; and
(c) at least one antibody.

2. The nanoconjugate of claim 1, wherein the ULV is comprised of dimyristoyl phosphatidylcholine (DMPC); dihexanoyl phosphatidylcholine (DHPC); dimyristoyl phosphatidylglycerol (DMPG); and distearoyl phosphoethanolamine-[maleimide(polyethylene glycol)-2000] (DSPE-PEG-maleimide).

3. The nanoconjugate of claim 1 or 2, wherein the contrast agent is a MRI contrast agent.

4. The nanoconjugate of claim 3, wherein the MRI contrast agent is gadolinium-diethylene-triamine-pentaacetic acid bis-oleate (Gd-DTPA-BOA).

5. The nanoconjugate of any one of claims 1 or 2, wherein the contrast agent is a radioisotope.

6. The nanoconjugate of any one of claims 1 or 2, wherein the contrast agent is a fluorophore.

7. The nanoconjugate of one of claims 3 or 4 further comprising a radioisotope contrast agent, or a fluorophore contrast agent, or both a radioisotope and a fluorophore.

8. The nanoconjugate of any one of claims 1 to 7, wherein the antibody specifically binds an epitope present in the brain endothelial cells.

9. The nanoconjugate of claim 8, wherein the antibody is specific for Insulin-like Growth Factor Binding Protein 7 (IGFBP7).

10. The nanoconjugate of claim 8, wherein the antibody comprises complementarity determining region (CDR) sequences RTSRRYAM (CDR1), GISRSGDGTHYAYSV (CDR2), and AAARTAFYYYGNDYNY (CDR3).

11. The nanoconjugate of claim 8, wherein the antibody comprises the sequence of SEQ ID NO. 5 or SEQ ID NO. 6., or a sequence substantially identical thereto.

12. The nanoconjugate of any one of claims 1 to 7, wherein the antibody is specific for EGFR.

13. The nanoconstruct of any one of claims 1 to 7, wherein the antibody comprises IgG C225.

14. A method of forming unilamellar vesicles (ULV) incorporating at least one contrast agent, the method comprising:
(a) mixing dimyristoyl phosphatidylcholine (DMPC); dihexanoyl phosphatidylcholine (DHPC); dimyristoyl phosphatidylglycerol (DMPG); distearoyl phosphoethanolamine-[maleimide(polyethylene glycol)-2000] (DSPE-PEG-maleimide) and gadolinium-diethylene-triamine-pentaacetic acid bis-oleate (Gd-DTPA-BOA); and
(b) allowing the spontaneous formation of ULV.

15. The method of claim 14, wherein, prior to step (a), an antibody is bioconjugated to DSPE-PEG-maleimide, thus incorporating the antibody into the nanoconjugate.

16. A method for in vivo imaging of cells or tissues in a mammal, the method comprising the steps of:
(a) administering to the mammal a composition comprising the nanoconjugate of any one of claims 1 to 7, wherein the antibody is specific for a selected receptor;
(b) waiting a time sufficient to allow the antibody to bind to the selected receptor; and
(c) imaging the cells or tissues with a non-invasive imaging technique whose resolution is enhanced by the presence of the nanoconjugates on or within the cells.

17. A method as recited in claim 16, wherein the imaging technique is selected from the group consisting of magnetic resonance imaging, magnetic spectroscopy, X-ray, positron emission tomography, optical imaging, computed tomography, and ultrasonic imaging.

18. The method of claim 16 or 17, wherein the selected receptor is specifically expressed by tumor endothelial cells.

19. The method of claim 16 or 17, wherein the selected receptor is IGFBP7.

20. The method of claim 19, wherein the single domain antibody comprises complementarity determining region (CDR) sequences RTSRRYAM or RTFSRLAM (CDR1), GISRSGDGTHYAYSV (CDR2), and AAARTAFYYYGNDYNY (CDR3).

21. The method of claim 19, wherein the single domain antibody comprises the sequence of SEQ ID NO. 5 or SEQ ID NO. 6., or a sequence substantially identical thereto.

22. The method of claim 16 or 17, wherein the selected receptor is EGFR.

23. The method of claim 22, wherein the antibody comprises IgG C225.

24. The method of any one of claims 18 to 23, wherein one or more tumors, metastases, vascularized malignant cell clusters, or individual malignant cells are imaged, selected from the group consisting of brain cancer, colon cancer, breast cancer, prostate cancer, lung cancer, pancreatic cancer, endometrial cancer, oral cancer, liver cancer, and renal cancer or any other cancer.

25. A method for detecting glioblastoma in a patient, comprising:
(a) contacting a tissue of interest with the nanoconjugate of any one of claims 8 to 13; and
(b) measuring the level of binding of the nanoconjugate, wherein an elevated level of binding, relative to normal tissue, is indicative that the tissue is neoplastic.

26. A method for detecting a tissue expressing IGFBP7, comprising:
(a) contacting a tissue of interest with the nanoconjugate of any one of claims 9 to 11; and
(b) measuring the level of binding of the nanoconjugate, wherein an elevated level of binding, relative to normal tissue is indicative of the presence of a tumor expressing IGFBP7.

27. A method for detecting a tissue expressing EGFR, comprising:
(a) contacting a tissue of interest with the nanoconjugate of claim 12 or 13; and
(b) measuring the level of binding of the nanoconjugate, wherein an elevated level of binding, relative to normal tissue is indicative of the presence of a tumor expressing EGFR.

28. The method of claim 26 or 27, wherein the step of measuring is performed by magnetic resonance imaging.

29. The method of any one of claims 26 to 28, wherein the nanoconjugate further comprises a fluorescent moiety and the step of measuring comprises fluorescence imaging.

30. A method for determining the location of glioblastoma brain tumor cells in a patient pre-operatively, intra-operatively, and/or post-operatively, comprising the steps of administering a composition comprising the nanoconjugate of one of claims 8 to 13 and a pharmaceutically acceptable carrier to the patient, wherein the composition is administered in an amount sufficient to image glioblastoma cells in vivo; and
(a) pre-operatively measuring the level of binding of nanoconjugate by magnetic resonance imaging to determine the location of glioblastoma cells, wherein an elevated level of binding, relative to normal tissue, is indicative of the presence of glioblastoma cells;
(b) intra-operatively measuring the level of binding of the nanoconjugate by fluorescence imaging to determine the location of residual glioblastoma cells, wherein an elevated level of binding, relative to normal tissue, is indicative of the presence of residual glioblastoma cells;
(c) post-operatively measuring the level of binding of the nanoconjugate by magnetic resonance imaging to determine the location of glioblastoma cells, wherein an elevated level of binding, relative to normal tissue, is indicative of the presence of tumor cells; or
(d) a combination of (a), (b) or (c) above.

31. A method for in vitro detection or quantification of biological or chemical molecule in a sample, the method comprising the steps of:
(a) contacting the sample with a solution comprising a nanoconjugate of any one of claims 1 to 6, so as to form a complex between the molecule and the particle; and
(b) detecting or quantifying said complex formed.

32. The method of claim 31, wherein the step of detecting or quantifying is performed by magnetic resonance imaging, optical imaging, or a combination thereof.


SEQUENCE LISTING (6 sequences)

SEQ ID NO: 1 - protein, 8 aa, Artificial Sequence
  Complementarity determining region sequence; CDR1
RTSRRYAM

SEQ ID NO: 2 - protein, 8 aa, Artificial Sequence
  Complementarity determining region sequence; CDR1
RTFSRLAM

SEQ ID NO: 3 - protein, 15 aa, Artificial Sequence
  Complementarity determining region sequence; CDR2
GISRSGDGTHYAYSV

SEQ ID NO: 4 - protein, 16 aa, Artificial Sequence
  Complementarity determining region sequence; CDR3
AAARTAFYYYGNDYNY

SEQ ID NO: 5 - protein, 140 aa, Artificial Sequence
  Single domain antibody specific to an epitope present in brain endothelial cells or receptor expressed by tumor endothelial cells
AIAIAVALAGFATVAQAQVKLEESGGGLVQAGGSLRLSCAASGRTSRRYAMGWFRQAPGKEREFVAGISRSGDGTHYAYSVKGRFTISRDNAANTVELQMNSLKPEDTAVYFCAAARTAFYYYGNDYNYWGQGTQVTVSS

SEQ ID NO: 6 - protein, 140 aa, Artificial Sequence
  Single domain antibody specific to an epitope present in brain endothelial cells or receptor expressed by tumor endothelial cells
AIAIAVALAGFATVAQAQVKLEESGGGSVQPGGSLRLSCAASGRTFSRLAMGWFRQAPGKERELVAGISRSGDGTHYAYSVKGRFTISRDNAANTVELQMNSLKPEDTAVYFCAAARTAFYYYGNDYNYWGQGTQVTVSS